(19) United States
(12) Patent Application Publication
(10) Pub. No.: US 2023/0064990 A1
(43) Pub. Date: Mar. 2, 2023

(54) DOSING REGIMENS AND METHODS FOR TREATING CANCER

(71) Applicant: Panbela Therapeutics, Inc., Waconia, MN (US)

(72) Inventors: Suzanne Gagnon, Jamestown, RI (US); Michael T. Cullen, Sanibel, FL (US); Thomas X. Neenan, Boston, MA (US); Michael Walker, Burnsville, MN (US)

(21) Appl. No.: 17/866,876

(22) Filed: Jul. 18, 2022

Related U.S. Application Data
(63) Continuation of application No. PCT/US21/14112, filed on Jan. 20, 2021.
(60) Provisional application No. 62/963,492, filed on Jan. 20, 2020.

(51) Int. Cl. A61K 31/131; A61K 31/133; A61K 31/337; A61K 31/7068; A61P 35/00

(52) U.S. Cl. CPC A61K 31/131; A61K 31/133; A61K 31/337; A61K 31/7068; A61P 35/00

(57) ABSTRACT

The invention provides methods for treating cancer in a patient comprising administering dosing regimens of (S,S)-(HO)2DEHSPM that unexpectedly reverse or reduce the onset of severe liver toxicity and improve patient safety profiles.

### RELATED APPLICATIONS

This application is a continuation of International Application No. PCT/US21/14112, which designated the United States and was filed on Jan. 20, 2021, published in English, which claims the benefit of U.S. Provisional Application No. 62/963,492, filed on Jan. 20, 2020. The entire teachings of the above applications are incorporated herein by reference.

### BACKGROUND OF THE INVENTION

Adenocarcinoma of the pancreas affects approximately 460,000 people worldwide annually including 55,440 in the United States (US) and 3,364 in Australia. It is the 3rd leading cause of death from cancer in the US. Pancreatic ductal adenocarcinoma (PDA) represents approximately 95% of all pancreatic cancers, with a 5-year survival rate of approximately 8.5%. Considering that the median overall survival for previously untreated patients with metastatic disease and good performance status is between 8.5 months and 11.1 months with the best available treatment regimens, effective treatment for PDA remains a major unmet medical need.

The diagnosis of pancreatic cancer is often delayed because the initial clinical signs and symptoms are vague and non-specific. By the time the diagnosis is made, approximately 85% of patients have locally advanced or metastatic tumors (usually to regional lymph nodes, liver, lung and peritoneum), and are therefore not amenable to surgical resection with curative intent. The most common presenting symptoms include weight loss, epigastric and/or back pain, and jaundice, sometimes in the setting of recent onset diabetes. The back pain is typically dull, constant, and of visceral origin radiating to the back, in contrast to the epigastric pain which is vague and intermittent. Less common symptoms include nausea, vomiting, diarrhea, anorexia, and glucose intolerance.

Currently, surgical resection offers the only potentially curative therapy, but since most patients have disease that is locally advanced or metastatic at the time of diagnosis, resection is infrequently an option. The prognosis for these patients is poor and most die from complications related to progression. The mainstay of treatment for metastatic disease is chemotherapy.

Current chemotherapy treatment regimens include single agent gemcitabine and various gemcitabine combinations to the multi-drug FOLFIRINOX (leucovorin (folinic acid), fluorouracil, irinotecan and oxaliplatin) regimen, which is frequently supplemented with white blood cell (WBC) growth factors. These treatments deliver to selected patients with good performance status median survival benefits ranging from 7 weeks to 4 months versus controls of gemcitabine alone. Clearly, more effective treatments for unresectable pancreatic ductal adenocarcinoma and other cancers are needed that also provide an improved patient safety profile.

### SUMMARY OF THE INVENTION

The invention provides methods for treating cancer in a patient comprising administering dosing regimens of (S,S)-(HO)2DEHSPM ((6S,15S)-3,8,13,18-teraazaicosane-6,15-diol), that unexpectedly reverse or reduce the onset of severe liver toxicities and improve patient safety profiles.

One preferred method of the invention comprises administering (S,S)-(HO)2DEHSPM, or a pharmaceutically acceptable salt thereof, as a daily dose on each of 5 consecutive days during the first two to four treatment cycles wherein each treatment cycle is about 28 days, optionally followed by one or more treatment cycles wherein (S,S)-(HO)2DEHSPM is administered periodically on days 1, 8 and 15 of each treatment cycle. Preferably, the method further includes the step of co-administering gemcitabine (also referred to herein as “GEM” or “G”), nab-paclitaxel (also referred to herein as “NAB” or “A”) or both during one or more treatment cycles. Preferably GEM and/or NAB are administered on days 1, 8, and 15 during each treatment cycle in which (S,S)-(HO)2DEHSPM is also being administered.

The invention also provides methods for reversing or reducing the onset of liver toxicities in a cancer in a patient treated with (S,S)-(HO)2DEHSPM comprising discontinuing dosing of (S,S)-(HO)2DEHSPM or reducing the dose of (S,S)-(HO)2DEHSPM by at least about 25% of the starting dose when liver toxicity becomes severe, followed by a return to dosing of (S,S)-(HO)2DEHSPM at about 50% of the starting dose for at least one or more treatment cycles administered until the liver toxicities are reversed.

### BRIEF DESCRIPTION OF THE DRAWINGS

FIG. 1 is a graph showing the best response per subject—Cohorts 2 and 3, N=13. Best response in evaluable subjects was PR in 8 (62%), SD in 5 (38%). Three subjects did not have post baseline scans with RECIST tumor assessments.

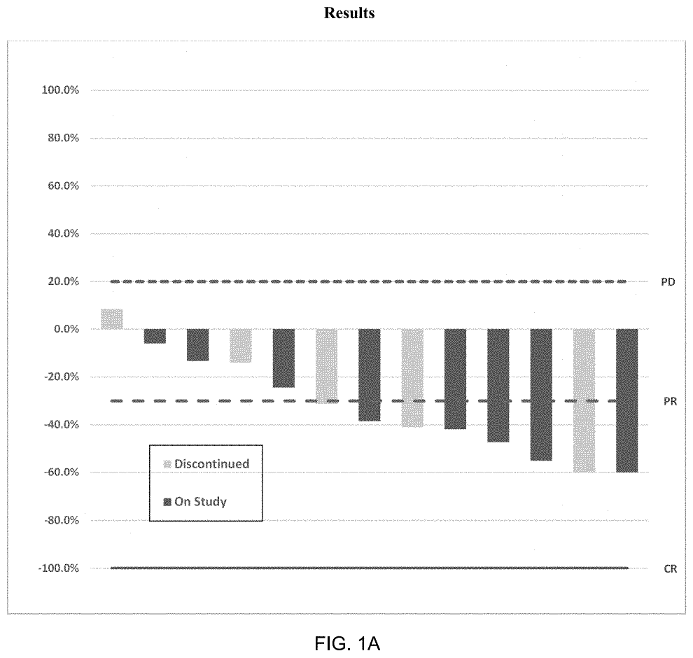

FIG. 1B is a graph showing the maximum CA19-9 percent change from baseline by response—cohorts 2 and 3, N=16. Eleven subjects in cohorts 2 and 3 (69%) had a CA 19-9 maximum decrease greater than 60%. ND—Not Done.

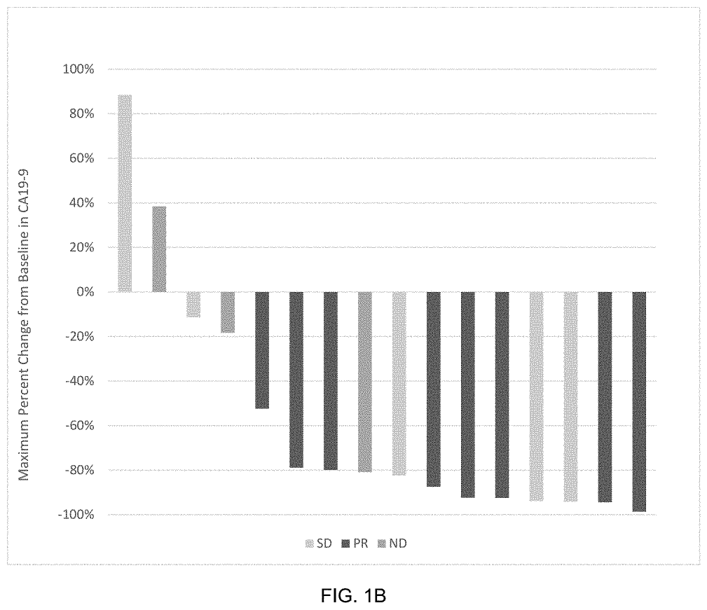

FIG. 1C is a graph showing days on study for Cohorts 2 and 3. As of Jan. 4, 2020, 8 of 16 subjects in cohorts 2 and 3 remain on study. Reasons for discontinuation include radiologic PD (N=1), adverse events (N=2), clinical progression (N=4), and patient decision (N=1). Four subjects have expired from pancreatic cancer.

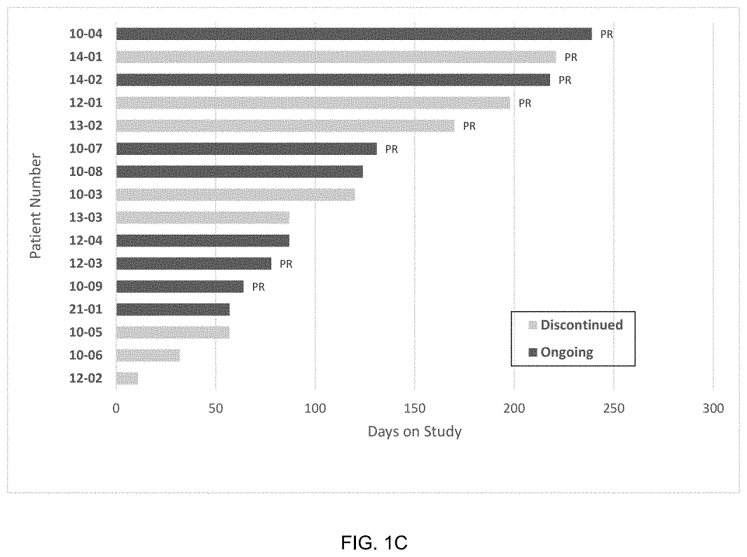

### DETAILED DESCRIPTION OF THE INVENTION

### Definitions

Those skilled in the art will recognize or be able to ascertain, using no more than routine experimentation, many equivalents to the specific embodiments in accordance with the invention described herein. The scope of the present invention is not intended to be limited to the following description, but rather is as set forth in the appended claims.

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

It is also noted that the term “comprising” is intended to be open and permits but does not require the inclusion of additional elements or steps. When the term “comprising” is used herein, the term “consisting of” is thus also encompassed and disclosed.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise.

As used herein, the term “about” or “approximately” as applied to a stated value, refers to a value that is within 10% of the stated value, that is, from 90% of the stated value to 110% of the stated value. In certain embodiments, the term “about” refers to a range of values that fall within 10%, 9%, 8%, 7%, 6%, 5%, 4%, 3%, 2%, 1%, or less in either direction (greater than or less than) of the stated value unless otherwise stated or otherwise evident from the context (except where such number would exceed 100% of a possible value).

As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena. Preferably pharmaceutically acceptable means approved or approvable by a regulatory agency of the Federal or a state government or the corresponding agency in countries other than the United States, or that is listed in the U.S. Pharmacopoeia or other generally recognized pharmacopoeia, for use in animals, and more particularly, in humans.

As used herein, the term “subject” or “patient” refers to any organism to which a composition in accordance with the present disclosure may be administered, e.g., for experimental, diagnostic, prophylactic, and/or therapeutic purposes. Typical subjects include animals (e.g., mammals such as mice, rats, rabbits, non-human primates, and humans) and/or plants. Preferably “patient” refers to a human subject who may seek or be in need of treatment, requires treatment, is receiving treatment, will receive treatment, or a subject who is under care by a trained professional for a particular disease or condition.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The term “pharmaceutically acceptable excipient” refers to a diluent, adjuvant, excipient or carrier with which a compound of the disclosure is administered. A pharmaceutically acceptable excipient is generally a substance that is non-toxic, biologically tolerable, and otherwise biologically suitable for administration to a subject, such as an inert substance, added to a pharmacological composition or otherwise used as a vehicle, carrier, or diluent to facilitate administration of an agent and that is compatible therewith. Examples of excipients include water, any and all solvents, dispersion media, diluents, or other liquid vehicles, dispersion or suspension aids, surface active agents, isotonic agents, thickening or emulsifying agents, preservatives, solid binders, lubricants and the like, as suited to the particular dosage form desired. Remington's The Science and Practice of Pharmacy, 21^{st} Edition, A. R. Gennaro (Lippincott, Williams & Wilkins, Baltimore, Md., 2006; incorporated herein by reference) discloses various excipients used in formulating pharmaceutical compositions and known techniques for the preparation thereof. Except insofar as any conventional excipient medium is incompatible with a substance or its derivatives, such as by producing any undesirable biological effect or otherwise interacting in a deleterious manner with any other component(s) of the pharmaceutical composition, its use is contemplated to be within the scope of this present disclosure.

As used herein any form of administration or coadministration of a “combination”, “combined therapy” and/or “combined treatment regimen” refers to at least two therapeutically active drugs or compositions which may be administered or co-administered”, simultaneously, in either separate or combined formulations, or sequentially at different times separated by minutes, hours or days, but in some way act together to provide the desired therapeutic response.

The term “therapeutic agent” encompasses any agent administered to treat a symptom or disease in an individual in need of such treatment. Such additional therapeutic agent may comprise any active ingredients suitable for the particular indication being treated, preferably those with complementary activities that do not adversely affect each other. Preferably, an additional therapeutic agent is an anti-inflammatory agent.

The term “chemotherapeutic agent” refers to a compound or a derivative thereof that can interact with a cancer cell, thereby reducing the proliferative status of the cell and/or killing the cell for example, by impairing cell division or DNA synthesis, or by damaging DNA, effectively targeting fast dividing cells. Examples of chemotherapeutic agents include, but are not limited to, alkylating agents (e.g., cyclophosphamide, oxaliplatin, ifosfamide); metabolic antagonists (e.g., methotrexate (MTX), 5-fluorouracil or derivatives thereof); a substituted nucleotide; a substituted nucleoside; DNA demethylating agents (also known as antimetabolites; e.g., azacitidine); antitumor antibiotics (e.g., mitomycin, adriamycin); plant-derived antitumor agents (e.g., irinotecan vincristine, vindesine, TAXOL®, paclitaxel, nab-pacitaxel, abraxane); cisplatin; carboplatin; etoposide; and the like. Such agents may further include, but are not limited to, the anti-cancer agents trimethotrexate (TMTX); temozolomide; raltitrexed; S-(4-Nitrobenzyl)-6-thioinosine (NBMPR); 6-benzyguanidine (6-BG); a nitrosoureas a nitrosourea (rabinopyranosyl-N-methyl-N-nitrosourea (Aranose), Carmustine (BCNU, BiCNU), Chlorozotocin, Ethylnitrosourea (ENU), Fotemustine, Lomustine (CCNU), Nimustine, N-Nitroso-N-methylurea (NMU), Ranimustine (MCNU), Semustine, Streptozocin (Streptozotocin)); cytarabine; and camptothecin; or a therapeutic derivative of any thereof. Chemotherapeutic agents also include chemotherapeutic cocktails such as FOLFIRINOX.

The phrase “therapeutically effective amount” or an “effective amount” refers to the administration of an agent to a subject, either alone or as part of a pharmaceutical composition and either in a single dose or as part of a series of doses, in an amount capable of having any detectable, positive effect on any symptom, aspect, or characteristic of a disease, disorder or condition when administered to the subject. The therapeutically effective amount can be ascertained by measuring relevant physiological effects, and it can be adjusted in connection with the dosing regimen and diagnostic analysis of the subject's condition, and the like. In reference to cancer or pathologies related to unregulated cell division, a therapeutically effective amount refers to that amount which has the effect of (1) reducing the size of a tumor (i.e. tumor regression), (2) inhibiting (that is, slowing to some extent, preferably stopping) aberrant cell division, for example cancer cell division, (3) preventing or reducing the metastasis of cancer cells, and/or, (4) relieving to some extent (or, preferably, eliminating) one or more symptoms associated with a pathology related to or caused in part by unregulated or aberrant cellular division, including for example, cancer.

An “effective amount” is also that amount that results in desirable PD and PK profiles and desirable immune cell profiling upon administration of the therapeutically active compositions of the invention.

As used herein, the term “parenteral” refers to dosage forms that are intended for administration as an injection or infusion and includes subcutaneous, intravenous, intra-arterial, intraperitoneal, intracardiac, intrathecal, and intramuscular injection, as well as infusion injections usually by the intravenous route.

The terms “treating” or “treatment” of a disease (or a condition or a disorder) as used herein refer to preventing the disease from occurring in a human subject or an animal subject that may be predisposed to the disease but does not yet experience or exhibit symptoms of the disease (prophylactic treatment), inhibiting the disease (slowing or arresting its development), providing relief from the symptoms or side-effects of the disease (including palliative treatment), and causing regression of the disease. With regard to cancer, these terms also mean that the life expectancy of an individual affected with a cancer may be increased or that one or more of the symptoms of the disease will be reduced. “Treating” also includes enhancing or prolonging an anti-tumor response in a subject.

As used herein, the term “preventing” refers to partially or completely delaying onset of an infection, disease, disorder and/or condition; partially or completely delaying onset of one or more symptoms, features, or clinical manifestations of a particular infection, disease, disorder, and/or condition; partially or completely delaying onset of one or more symptoms, features, or manifestations of a particular infection, disease, disorder, and/or condition; partially or completely delaying progression from an infection, a particular disease, disorder and/or condition; and/or decreasing the risk of developing pathology associated with the infection, the disease, disorder, and/or condition.

The phrase “causing chemical resection or ablation of the function of the entire exocrine portion of the pancreas” as used herein refers to the elimination of substantially all function of the exocrine portion of the pancreas and includes eliminating a clinically significant number of acinar cells in the exocrine portion of the pancreas, and/or physical shrinkage of the pancreas to less than 30% of the original size.

The term RECIST stands for Response Evaluation Criteria in Solid Tumors is a set of rules established and published by a collaboration of international authorities (e.g., European Organization for Research and Treatment of Cancer (EORTC), National Cancer Institute (NCI) of the U.S. and National Cancer Institute of Canada) that define when cancer patients improve (“respond”), stay the same (“stable”) or worsen (“progression) during treatments.

“Progression free survival (PFS),” as used in the context of the cancers described herein, refers to the length of time during and after treatment of the cancer until objective tumor progression or death of the patient. The treatment may be assessed by objective or subjective parameters; including the results of a physical examination, neurological examination, or psychiatric evaluation. In preferred aspects, PFS may be assessed by blinded imaging central review and may further optionally be confirmed by ORR or by blinded independent central review (BICR).

“Overall survival (OS)” may be assessed by OS rate at certain time points (e.g., 1 year and 2 years) by the Kaplan-Meier method and corresponding 95% CI will be derived based on Greenwood formula by study treatment for each tumor type. OS rate is defined as the proportion of participants who are alive at the time point. OS for a participant is defined as the time from the first dosing date to the date of death due to any cause.

As used herein a “complete response” is the disappearance of all signs of cancer in response to treatment. A complete response may also be referred to herein as “total remission”.

As used herein the term “partial response” means a decrease in the size of the tumor, or in the extent of cancer in the body in response to treatment. A partial response may also be referred to herein as “partial remission”.

The term “cancer”, as used herein, shall be given its ordinary meaning, as a general term for diseases in which abnormal cells divide without control.

The term “reducing a tumor” or “tumor regression” as used herein refers to a reduction in the weight, size or volume of a tumor mass, a decrease in the number of metastasized tumors in a subject, a decrease in the proliferative status (the degree to which the cancer cells are multiplying) of the cancer cells. For example, the weight, size or volume of a tumor may be reduced by about 1%, 5%, 10%, 15%, 20%, 25%, 30%, 35%, 40%, 45%, 50%, 55%, 60%, 65%, 70%, 75%, 80%, 85%, 90%, 95% or 98% or more as compared to baseline. Techniques for establishing whether a tumor has been reduced or regressed are known in the art.

As used herein, “total cumulative dose” (TCD) of (5, S)-(HO)2DEHSPM in any dosing regimen, refers to the total amount (S,S)-(HO)2DEHSPM dosed in a patient at a specified dose for a specified period of time. In the context of the present invention TCD preferably refers to the total dose of (S,S)-(HO)2DEHSPM administered to the patient during a single treatment cycle or after all treatment cycles are complete.

The term “treatment cycle” has its usual meaning in the art with respect to chemotherapy and refers to a course of administration of a chemotherapeutic drug followed by a period of time when no drug is administered. The treatment period with the drug and the rest period combine to make up one treatment cycle. Unless otherwise specified, the term “treatment cycle” as used herein refers to a 28-day treatment cycle.

As used herein “Grade 3” or “severe” liver toxicity is determined based on standardized definitions for adverse events (AEs) that occur during human clinical trials as established by the National Cancer Institute (NCI). The National Cancer Institute (NCI) of the National Institutes of Health (NIH) has published standardized definitions for adverse events (AEs), known as the Common Terminology Criteria for Adverse Events (CTCAE), also called “common toxicity criteria” (CTC), to describe the severity of organ toxicity for patients receiving cancer therapy. In CTCAE, an adverse event (AE) is defined as any abnormal clinical finding temporally associated with the use of a therapy for cancer; causality is not required. These criteria are used for the management of chemotherapy administration and dosing, and in clinical trials to provide standardization and consistency in the definition of treatment-related toxicity. For liver toxicities, the CTCAE has classified elevations of serum enzyme activities (alanine aminotransferase” (ALT) and aspartate aminotransferase (AST)) into mild (grade 1) if >ULN (upper limits of normal) to 3×ULN; moderate (grade 2) if >3 to 5×ULN; severe (grade 3) if >5 to 20×ULN; and life-threatening (grade 4) if >20×ULN; and with no definition for fatal (grade 5). Similarly, they graded serum total bilirubin concentration as mild if >ULN to 1.5×ULN, moderate if >1.5 to 3×ULN, severe if >3 to 8×ULN, and life-threatening if >8×ULN.

### (S,S)-(HO)2DEHSPM Drug Product

(HO)2DEHSPM is a small molecule, ethylated and hydroxylated derivative of homospermine, a polyamine analogue similar to endogenous spermine and has the following formula 1.

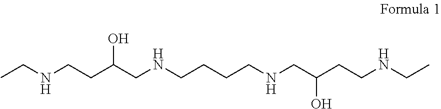 [SMILES: CCNCCC(O)CNCCCCNCC(O)CCNCC]

It will be appreciated by those skilled in the art that the compound of Formula 1 contains at least two chiral centers. The compound of Formula 1 may exist in the form of two different optical isomers (i.e. (+) or (−) enantiomers) and a diastereomer. All such enantiomers, diastereomers and mixtures thereof including racemic mixtures are included within the scope of the invention. The enantiomers of the compound of Formula 1 can be obtained by methods disclosed in U.S. Pat. No. 6,160,022 and WO 2019/152323. The enantiomers of the compound of Formula 1 can also be obtained from a racemic mixture by methods well known in the art, such as chiral HPLC and chemical resolution. Alternatively, the enantiomers of the compound of Formulas 1 can be synthesized by using optically active starting materials.

(S,S)-(HO)2DEHSPM is the S,S enantiomer of Formula 1. The chemical name for (S,S)-(HO)2DEHSPM is (6S,15S)-3,8,13,18-teraazaicosane-6,15-diol or N¹,N¹⁴-diethyl-3S,12S-dihydroxyhomospermine and may also be referred to herein (HO)2DEHSPM). The CAS number for (S,S)-(HO)2DEHSPM is 259657-09-5. The compound is preferably isolated, formulated and administered in the form of a pharmaceutically acceptable salt, and all reference to (S,S)-(HO)2DEHSPM in relation to compositions and administration refers to free base and salt forms unless otherwise stated. The preferred form of (S,S)-(HO)2DEHSPM is the stable tetrahydrochloride salt, referred to herein as (S,S)-(HO)2DEHSPM.4HCl. All references to doses of (S,S)-(HO)2DEHSPM (for example, in units of mg, mg/kg or mg/kg/day) herein refer to the mass of (S,S)-(HO)2DEHSPM.4HCl unless otherwise specified. In certain instances, the (S,S)-(HO)2DEHSPM.4HCl dose is followed by the corresponding free base equivalent dose in parentheses. For example, reference to a dose of “0.4 mg/kg/day (0.27 mg/kg/day)” refers to a dose of (S,S)-(HO)2DEHSPM.4HCl of 0.4 mg/kg/day and the corresponding free base equivalent dose of 0.27 mg/kg/day.

Polyamines (PA) including spermine are ubiquitous biological molecules found in all mammalian cells. Polyamines are essential for the growth, reproduction and function of normal cells, and programmed cell death (apoptosis). Each of the three native polyamines (spermine, spermidine and putrescine) are metabolized intracellularly with levels maintained within narrow ranges by a series of enzymes including ornithine decarboxylase (ODC), S-adenosylmethione decarboxylase (SAMDC), spermidine/spermine N1-acetyltransferase (SSAT), polyamine oxidase (PAO) and others. Polyamine metabolism via SSAT and PAO generates hydrogen peroxide (H₂O₂), which induces SSAT and apoptosis, and may, if unchecked, lead to a positive cell-death-signal-generating cycle.

Increased biosynthesis of polyamines and their biosynthetic enzymes in neoplastic tissues has made this class of molecules a promising target for cancer therapeutic efforts. The polyamine transport uptake mechanism appears to be up regulated in various tumor types, including pancreatic ductal adenocarcinoma where demand for polyamines is high.

Inducing polyamine depletion via the cellular uptake of dysfunctional synthetic polyamine analogues has been proposed as an antitumor strategy. Polyamine analogues enter cells via polyamine transporters, substitute for natural polyamines in their self-regulatory roles, but fail to function as natural polyamines in promoting cell growth. Consequently, a state of “pseudo-polyamine” excess is created in cells, thereby downregulating the enzymes responsible for polyamine synthesis, and in some cases inducing SSAT, the key enzyme responsible for intracellular polyamine catabolism.

(S,S)-(HO)2DEHSPM is a dysfunctional analogue of the naturally occurring polyamine spermine. It inhibits cell growth by substituting for spermine, reduces spermine levels and depletes intracellular pools of spermidine and putrescine. This strategy may be useful against many types of cancer, for example solid tumors.

Administration of (S,S)-(HO)2DEHSPM was found to be effective in decreasing tumor burden in three different murine xenograft models of human pancreatic adenocarcinoma. Antineoplastic effects of (S,S)-(HO)2DEHSPM were further demonstrated in in vitro cell viability studies using six human pancreatic tumor cell lines.

Preclinical animal data with (S,S)-(HO)2DEHSPM suggest that both efficacy against pancreatic tumor cells and ablation of the beagle exocrine pancreas are cumulative effects of (S,S)-(HO)2DEHSPM not requiring high plasma drug levels, but rather a total cumulative dose (TCD). The first-in-human Phase 1 study was conducted to determine the maximum tolerated dose (MTD) and dose limiting toxicities (DLTs) of (S,S)-(HO)2DEHSPM in patients with previously treated locally advanced or metastatic pancreatic ductal adenocarcinoma. The dosing schedule in the first-in-human Phase 1 study was selected using the effective dose for exocrine pancreas ablation as a surrogate for anti-tumor effect.

In addition to neoplastic tissues, the acinar cells of the exocrine pancreas also appear to exhibit enhanced uptake of polyamines compared to other tissues, as evidenced by high pancreas tissue levels post-exposure to (HO)2DEHSPM enantiomers. Cumulative exposure to repeat doses of (S,S)-(HO)2DEHSPM in healthy beagles resulted in exocrine pancreatic atrophy with exocrine pancreatic insufficiency without an inflammatory response and with preservation of islet cell function. This effect on the exocrine pancreas in dogs (causing nearly complete ablation of the exocrine pancreas) was an unexpected finding in development of this polyamine analogue as a therapeutic agent. It occurred in a dose-dependent manner several weeks after drug dosing was discontinued in dogs. Beginning 5 to 6 weeks post last dose, the animals at the high-dose levels rapidly lost weight and their serum trypsin-like immunoreactivity decreased to <2.5 μg/L, which is diagnostic for exocrine pancreatic insufficiency in this species. In addition, fat absorption tests became abnormal and hepatic transaminase values increased. The pancreata of euthanized animals were grossly atrophied with diffuse moderate to severe pancreatic atrophy, especially of the acini. However, the islets appeared both histologically intact and functional based on serum glucose levels and oral glucose tolerance tests.

### Pharmaceutical Compositions

(S,S)-(HO)2DEHSPM or a pharmaceutically acceptable salt thereof, is preferably formulated as a pharmaceutical composition with one or more pharmaceutically acceptable diluents, carriers or excipients. The pharmaceutical compositions are preferably formulated for administration to a patient by injection, preferably, parenteral injection and even more preferably by subcutaneous injection. Preferably (S,S)-(HO)2DEHSPM is formulated in the form of (S,S)-(HO)2DEHSPM.4HCl, for example, in a clear sterile solution in pH-adjusted sterile water for injection, preferably subcutaneous injection. However, other modes of administration of (S,S)-(HO)2DEHSPM or a pharmaceutically acceptable salt thereof are also contemplated, such as oral, pulmonary, nasal, buccal, rectal, sublingual and transdermal.

### Dosing Regimens

For the first-in-human (Phase 1a/1b) study, a starting dose of 0.05 mg/kg (1.8 mg/m²) of (S,S)-(HO)2DEHSPM.4HCl was chosen as 1/10 the severely toxic dose (STD₁₀) observed in a 4-week repeated dose toxicity study in rats (3 mg/kg/day; 1.8 mg/m²). This dose was chosen in accordance with the ICH S9 Guideline for the Nonclinical Development for Anti-Cancer Pharmaceuticals. For the Phase 1a/1b dosing schedule patients were administered up to 0.4 mg/kg/day Monday through Friday for 3 weeks for a total cumulative dose (TCD) of 6 mg/kg. In this animal experiment, one cycle consisted of 3 weeks of dosing and 5 weeks of rest for a total cycle length of 8 weeks.

The Phase 1a/1b dosing schedule was designed to evaluate both individual dose levels as well as total cumulative dose. Although it is known that many human tumors have accelerated polyamine uptake mechanisms, and destruction of pancreatic tumors were expected to occur prior to any off-target effects in normal tissue (e.g., acinar cell atrophy), the dosing schedule was intended to evaluate a sufficient cumulative dose of (S,S)-(HO)2DEHSPM to produce an anti-tumor effect even if this exposure may cause effects in non-tumor tissue.

The maximum total cumulative dose (TCD) that was planned to be administered in the initial Phase 1a/1b study was 60 mg/kg, which is the human equivalent of the mean highest cumulative dosages administered to nude mice in xenograft studies (Example 2) that produced significant tumor reduction at the maximum tolerated dose (MTD). During the dose escalation phase of study (Phase 1a, Example 3), the maximum tolerated dose of (S,S)-(HO)2DEHSPM was determined to be less than 0.8 mg/kg/day. No drug-related serious adverse events (SAEs) or dose limiting toxicities (DLTs) were observed in subjects receiving up to 0.4 mg/kg/day Monday through Friday×3 weeks (TCD˜6 mg/kg per 8 week cycle).

During preclinical studies with nude mice and beagle dogs, it was discovered that minimum effective cumulative dosages were demonstrated where “effective” was defined as the dosages and duration of treatment that resulted in either significant reduction in tumor volume including metastases (mice) or atrophy of normal exocrine pancreatic cells (dogs). “Minimum” was defined as those dosages and durations that were effective with the least number of adverse findings or changes in any organ system other than the canine exocrine pancreas or human pancreatic tumor cells. During preclinical studies it was also discovered that both efficacy against pancreatic tumor cells and ablation of exocrine pancreas are cumulative effects of (S,S)-(HO)2DEHSPM not requiring high plasma drug levels, but rather a total cumulative dose (TCD). Therefore, unlike other drugs which rely on dosing to achieve specific plasma concentration for efficacy, effective dosing of (S,S)-(HO)2DEHSPM requires a delicate balance to determine minimum effective TCD for tumor reduction but that that avoids off target effects and liver toxicity.

Additional data from the Phase 1a/1b study (Example 4) showed elevated liver toxicities in several patients with a rating of “severe” or Grade 3 in accordance with the Common Terminology Criteria for Adverse Events (CTCAE) established for clinical trials by NCI. In order to mitigate liver toxicity, but maintain the minimum effective TCD, it was discovered that a modified daily dosing regimen referred to herein as “shortened daily dosing regimen” during each 28 day treatment cycle unexpectedly improves toxicity profiles particularly liver toxicities in patients as compared to a reference dosing schedule. A “reference dosing schedule” as that term is used herein refers to the following dosing schedule: daily dosing of (S,S)-(HO)2DEHSPM for 5 consecutive days (e.g., days 1-5) of each treatment cycle for at least three consecutive treatment cycles and preferably at least 5 consecutive treatment cycles and more preferably at least 8 consecutive treatment cycles and wherein each treatment cycle is 28 days.

A “shortened daily dosing regimen” may comprise, for example, daily dosing for 5 consecutive days (e.g., days 1-5) of each treatment cycle for no more than 5 consecutive treatment cycles, preferably no more than 4 consecutive treatment cycles, preferably no more than 3 consecutive treatment cycles, preferably no more than 2 consecutive treatment cycles and preferably no more than 1 treatment cycle wherein a treatment cycle is 28 days. Preferably a shortened daily dosing regimen is used for no more than 2 consecutive treatment cycles and preferably for only 1 treatment cycle wherein (S,S)-(HO)2DEHSPM is dosed daily for no more than 5 consecutive days of each treatment cycle (e.g., days 1-5).

The amount of each dose of (S,S)-(HO)2DEHSPM in any treatment regimen of the invention may be the same as that which would have been delivered during a reference dosing schedule or may be a dose which is less than that which is used during a reference dosing schedule, for example a dose of (S,S)-(HO)2DEHSPM which is reduced by 1%, 5%, 10%, 15%, 20%, 25%, 30%, 35%, 40%, 45%, 50%, 55%, 60%, 65%, 70%, 75%, 80%, 85%, 90%, 95% or 98% or more. Preferred doses of (S,S)-(HO)2DEHSPM include, but are not limited to the following doses, 0.2 mg/kg/day (0.14 mg/kg/day), 0.4 mg/kg/day (0.27 mg/kg/day), and 0.6 mg/kg/day (0.41 mg/kg/day).

Another preferred dosing regimen of the invention comprises a combination of a shortened daily dosing regimen of (S,S)-(HO)2DEHSPM with periodic dosing of (S,S)-(HO)2DEHSPM in the same or different treatment cycles. This treatment regimen involving combining daily dosing of (S,S)-(HO)2DEHSPM with periodic dosing of (S,S)-(HO)2DEHSPM is referred to herein as a “combination shortened daily dosing regimen/periodic dosing regimen(s)”.

One preferred combination shortened daily dosing regimen/periodic dosing regimen comprises administering a shortened daily dosing regimen of (S,S)-(HO)2DEHSPM as described above followed by one or more treatment cycles wherein (S,S)-(HO)2DEHSPM is administered periodically, for example on days 1, 8 and 15 of each of the following treatment cycles wherein each treatment cycle is 28 days. Preferably, the shortened daily dosing regimen is administered for no more than 2 consecutive treatment cycles (e.g., cycles 1 and 2 only) followed by one or more treatment cycles (e.g., Cycles 3 to 8 or more) wherein (S,S)-(HO)2DEHSPM is administered periodically, for example on days 1, 8 and 15 of the treatment cycle and wherein each treatment cycle is 28 days. In one embodiment, (S,S)-(HO)2DEHSPM is administered to a patient on a shortened daily dosing regimen/periodic dosing regimen (S,S)-(HO)2DEHSPM at a dose of about 0.4 mg/kg (about 0.27 mg/kg) per treatment day. In other embodiments, (S,S)-(HO)2DEHSPM is administered to a patient on a shortened daily dosing regimen/periodic dosing regimen at a dose of about 0.4 mg/kg (about 0.27 mg/kg) per treatment day during the shortened daily dosing regimen and a dose of about 0.3 mg/kg (about 0.21 mg/kg) to about 0.5 mg/kg (about 0.34 mg/kg) per treatment day during the periodic dosing regimen. In certain embodiments, (S,S)-(HO)2DEHSPM is administered to the patient at a dose from about 0.35 mg/kg (about 0.24 mg/kg) to about 0.45 mg/kg (about 0.31 mg/kg) per treatment day during the periodic dosing regimen. Preferably, (S,S)-(HO)2DEHSPM is administered to the patient at a dose of about 0.4 mg/kg (about 0.27 mg/kg) per treatment day during the periodic dosing regimen. The patient can be treated for at least 2, at least 3, at least 4, at least 5, at least 6, at least 8, or at least 10 or more treatment cycles, or until a complete or partial response, disease progression or unacceptable toxicity occurs. In one embodiment, the patient is treated for two treatment cycles of the shortened daily dosing regimen followed by 4 to 6 treatment cycles of the periodic dosing regimen.

In certain embodiments of the dosing regimen of the invention, such as a shortened daily dosing regimen, a periodic dosing regimen or a shortened daily dosing regimen/periodic dosing regimen as disclosed above, is continued until the patient has received a pre-specified total cumulative dose (“TCD”) of (S,S)-(HO)2DEHSPM. In certain embodiments, the TCD is about 12 mg/kg (about 8.2 mg/kg) or less or about 10 mg/kg (about 6.9 mg/kg) or less. In certain embodiments, the TCD is from about 5 mg/kg (about 3.4 mg/kg) to about 12 mg/kg (about 8.2 mg/kg), about 5 mg/kg (about 3.4 mg/kg) to about 10 mg/kg (about 6.9 mg/kg), about 8 mg/kg (about 5.5 mg/kg) to about 10 mg/kg (about 6.9 mg/kg), about 8.5 mg/kg (about 5.8 mg/kg) to about 9.5 mg/kg (about 6.5 mg/kg). Preferably, the TCD is about 8.6 mg/kg (about 5.9 mg/kg) to about 9.0 mg/kg (about 6.2 mg/kg) or about 8.8 mg/kg (about 6.0 mg kg).

Another preferred dosing regimen of the invention comprises only periodic dosing of (S,S)-(HO)2DEHSPM for one or more treatment cycles (i.e. no daily dosing of (S,S)-(HO)2DEHSPM). This treatment regimen is referred to herein as a “periodic dosing only regimen(s)”. Preferred periodic dosing only regimens include dosing (S,S)-(HO)2DEHSPM periodically for no more than about 5 to no more than about 14 doses per treatment cycle wherein dosing occurs on non-consecutive days. One preferred periodic dosing regimen includes administering (S,S)-(HO)2DEHSPM on days 1, 8 and 15 of each of any one or more treatment cycles and preferably for at least 1, 2, 5, 8 or more treatment cycles. Another preferred periodic dosing regimen includes dosing (S,S)-(HO)2DEHSPM periodically for no more than about 5 to no more than about 10 doses for the first and second treatment cycles wherein dosing occurs on non-consecutive days and thereafter administering (S,S)-(HO)2DEHSPM on days 1, 8 and 15 of all treatment cycles thereafter (e.g., cycles 3 through 5 or more).

Another preferred dosing regimen is intended to reverse or mitigate liver toxicity during treatment as compared to, for example, liver toxicity associated with the reference dosing schedule. This dosing regimen is referred to herein as a “rescue dosing regimen” and comprises reducing the amount or frequency of dosing with (S,S)-(HO)2DEHSPM (including discontinuing dosing with (S,S)-(HO)2DEHSPM entirely) for a period of time for all or a part of one or more treatment cycles followed by resuming treatment with a dose of (S,S)-(HO)2DEHSPM at a dose which is less than that which is used during a reference dosing schedule such as a dose of (S,S)-(HO)2DEHSPM that is reduced by, for example about 1%, 5%, 10%, 15%, 20%, 25%, 30%, 35%, 40%, 45%, 50%, 55%, 60%, 65%, 70%, 75%, 80%, 85%, 90%, 95% or 98% or more.

The dosing regimens of the invention unexpectedly prevent severe liver toxicity while maintaining a minimally effective TCD as compared to, for example, the reference dosing schedule. Preferably the dosing schedules of the invention prevent severe liver toxicity of Grade 3 or higher in accordance with CTCAE established for clinical trials by NCI.

Therefore, it is understood that any of the above-described dosing schedules may be combined in various ways so that the TCD is effective to reduce the target tumor while also reducing liver toxicity by at least about 1%, 5%, 10%, 15%, 20%, 25%, 30%, 35%, 40%, 45%, 50%, 55%, 60%, 65%, 70%, 75%, 80%, 85%, 90%, 95% or 98% or more, particularly as compared to the reference dosing schedule. For example, a patient may be administered a shortened daily dosing regimen, but if it appears that the patient is experiencing severe liver toxicity of Grade 3 or higher, the patient may then be administered a rescue dosing regimen for one or more cycles and then may resume the originally shortened daily dosing regimen for one or more additional cycles, or alternatively, the combined shortened daily dosing regimen/periodic dosing regimen or alternatively the periodic only dosing regimen.

It is also understood that any one of the dosing regimens of the invention may be combined with the reference dosing schedule. For example, the reference dosing schedule may be used for cycles 1 and 2 followed by the rescue dosing regimen for one or more of cycles followed by the shortened daily dosing regimen and/or the combined shortened daily dosing regimen and/or the periodic dosing only regimen for one or more cycles.

In addition to reducing liver toxicities, the dosing regimens of the invention also unexpectedly reduce other side effects in patients including, but not limited to decreased gastrointestinal motility and pancreatic atrophy and insufficiency while achieving a minimally effective TCD. Preferably, the dosing regimens of the invention result in one or more of the following: reduced levels of polyamines e.g., putrescine, spermine, and spermidine in targeted cancer cells; inhibition of tumor growth; inhibition of metastases to other organs of the patient and inhibition of the growth of such mestatases.

It is understood that any of the dosing schedules of the invention may be carried out for 1 or more treatment cycles, such as 1 or more 28-day treatment cycles. Preferably a patient is treated for at least 2, preferably at least 3, preferably at least 4, preferably at least 5, preferably at least 6, preferably at least 8, and preferably at least 10 or more treatment cycles, or until a complete or partial response, disease progression or unacceptable toxicity occurs.

Combination Therapy with Gemcitabine (GEM) and Nab-Paclitaxel (NAB)

Preferably (S,S)-(HO)2DEHSPM is administered in combination with one or both of GEM and NAB. Preferably GEM and/or NAB is administered in a separate composition from (S,S)-(HO)2DEHSPM prior to, subsequent to, or simultaneously with (S,S)-(HO)2DEHSPM. Preferably, GEM is administered at a dose of about 1000 mg/m² and NAB is administered at a dose of 125 mg/m² or as per the standard prescribing recommendations, for one or more days of each treatment cycles, such as, for example, on days 1, 8, and 15 of a 28 day treatment cycle. Preferably GEM and NAB are administered together and the administration of both chemotherapeutics is referred to herein as administration of “GEM/NAB”.

Preferably, when co-administering (S,S)-(HO)2DEHSPM with GEM and/or NAB, (S,S)-(HO)2DEHSPM is administered according to any one of the dosing regimens of the invention described above, e.g., a shortened daily dosing regimen, a combined shortened daily dosing regimen/periodic dosing regimen or a periodic dosing regimen, for one or more treatment cycles and GEM/NAB and NAB are administered on days 1, 8, and 15 of one or more of the treatment cycles. Preferably during those treatment cycles wherein (S,S)-(HO)2DEHSPM is scheduled to be dosed on the same day as GEM and/or NAB, (S,S)-(HO)2DEHSPM is administered in a separate composition prior to administration of GEM and/or NAB. (S,S)-(HO)2DEHSPM may be administered minutes or hours before GEM and/or NAB.

In certain embodiments, the combination therapy comprises administering (S,S)-(HO)2DEHSPM for 5 consecutive days, such as days 1-5, during the first week of treatment cycles 1 and 2, and GEM/NAB on days 1, 8 and 15 of treatment cycles 1 and 2 (wherein each treatment cycle is 28 days), followed by co-administering (S,S)-(HO)2DEHSPM and GEM/NAB on days 1, 8 and 15 during cycle 3 and all cycles thereafter.

One preferred combination therapy comprises administering (S,S)-(HO)2DEHSPM on days 1-5 of treatment cycles 1 and 2 at a dose of about 0.4 mg/kg (about 0.27 mg/kg) per treatment day and GEM/NAB on days 1, 8 and 15 of treatment cycles 1 and 2 (wherein each treatment cycle is 28 days) followed by administering (S,S)-(HO)2DEHSPM at a dose of about 0.35 mg/kg (about 0.24 mg/kg) to about 0.45 mg/kg (about 0.31 mg/kg) per treatment day during the periodic dosing regimen or about 0.4 mg/kg (about 0.27 mg/kg) per treatment day during the periodic dosing regimen. GEM/NAB is preferably dosed on days 1, 8 and 15 of cycle 3 and all cycles thereafter. Preferably, the combination therapy is continued until a pre-determined TCD of (S,S)-(HO)2DEHSPM is reached as disclosed above. In certain embodiments, the TCD is about 12 mg/kg (about 8.2 mg/kg) or less or about 10 mg/kg (about 6.9 mg/kg) or less. In certain embodiments, the TCD is from about 5 mg/kg (about 3.4 mg/kg) to about 12 mg/kg (about 8.2 mg/kg), about 5 mg/kg (about 3.4 mg/kg) to about 10 mg/kg (about 6.9 mg/kg), about 8 mg/kg (about 5.5 mg/kg) to about 10 mg/kg (about 6.9 mg/kg), about 8.5 mg/kg (about 5.8 mg/kg) to about 9.5 mg/kg (about 6.5 mg/kg). Preferably, the TCD is about 8.6 mg/kg (about 5.9 mg/kg) to about 9.0 mg/kg (about 6.2 mg/kg) or about 8.8 mg/kg (about 6.0 mg kg).

Preferably (S,S)-(HO)2DEHSPM, in combination with one or both of GEM and NAB to treat and/or prevent various cancers serves to minimize any adverse effects associated with administration of the individual therapies by themselves. By way of example, the addition of (S,S)-(HO)2DEHSPM using a shortened daily treatment regimen, a combination shortened daily treatment regimen/periodic dosing regimen, a periodic dosing only regimen or a rescue dosing regimen in combination with GEM and/or NAB may allow a reduction of the amount of GEM or NAB needed to achieve the therapeutic goal, thus reducing (or even eliminating) severe and fatal adverse reactions associated with GEM and NAB.

For example, the similar toxicity profiles of GEM and NAB, including bone marrow suppression, fatigue and constitutional symptoms, and peripheral neuropathy (nab-paclitaxel), often require dose reductions or discontinuation of one or both drugs. Preclinical and clinical testing of (S,S)-(HO)2DEHSPM monotherapy showed that neither bone marrow suppression nor peripheral neuropathy were observed (Example 3), suggesting that these toxicities are unlikely to be exacerbated by treatment with a combination of (S,S)-(HO)2DEHSPM, GEM, and NAB. Thus, (S,S)-(HO)2DEHSPM administered in combination with GEM and NAB provides an effective alternative to treatment with standard chemotherapy with unexpected synergies in tumor reduction and regression.

The combination treatment regimens of (S,S)-(HO)2DEHSPM and of the invention are preferably administered for one or more cycles, to the patient until the patient is cured or until the patient is no longer benefiting from the treatment regimen.

### Additional Complementary Combination Therapies

While (S,S)-(HO)2DEHSPM dosing regimens of the invention may be used as a monotherapy or as combination therapy with GEM/NAB in accordance with the invention, the combination of dosing regimens of the invention with other anticancer treatments in the context of the invention is also contemplated. Examples of additional anticancer treatments that may be combined with dosing regimens of the invention as (S,S)-(HO)2DEHSPM monotherapy or further combined with the dosing regimens of the invention as (S,S)-(HO)2DEHSPM/GEM/NAB combination therapy, include the following anti-cancer therapies.

### Additional Cytotoxic and Chemotherapeutic Agents

Preferably, the methods of the invention include administration of (S,S)-(HO)2DEHSPM monotherapy or (S,S)-(HO)2DEHSPM/GEM/Nab combination therapy in further combination with administration with other cytotoxic/chemotherapeutic agents including but not limited to, alkylating agents, antitumor antibiotics, antimetabolic agents, other anti-tumor antibiotics, and agents derived from plants and other natural sources.

Alkylating agents are drugs which impair cell function by forming covalent bonds with amino, carboxyl, sulfhydryl and phosphate groups in biologically important molecules. The most important sites of alkylation are DNA, RNA and proteins. Alkylating agents depend on cell proliferation for activity but are not cell-cycle-phase-specific. Alkylating agents suitable for use in the present invention include, but are not limited to, bischloroethylamines (nitrogen mustards, e.g., chlorambucil, cyclophosphamide, ifosfamide, mechlorethamine, melphalan, uracil mustard), aziridines (e.g., thiotepa), alkyl alkone sulfonates (e.g., busulfan), nitroso-ureas (e.g., BCNU, carmustine, lomustine, streptozocin), nonclassic alkylating agents (e.g., altretamine, dacarbazine, and procarbazine), and platinum compounds (e.g., carboplastin, oxaliplatin and cisplatin).

Antitumor antibiotics like adriamycin intercalate DNA at guanine-cytosine and guanine-thymine sequences, resulting in spontaneous oxidation and formation of free oxygen radicals that cause strand breakage. Other antibiotic agents suitable for use in the present invention include, but are not limited to, anthracyclines (e.g., doxorubicin, daunorubicin, epirubicin, idarubicin and anthracenedione), mitomycin C, bleomycin, dactinomycin, and plicatomycin.

Antimetabolic agents suitable for use in the present invention include but are not limited to, floxuridine, fluorouracil, methotrexate, leucovorin, hydroxyurea, thioguanine, mercaptopurine, cytarabine, pentostatin, fludarabine phosphate, cladribine, and asparaginase.

Plant derived agents include taxanes, which are semisynthetic derivatives of extracted precursors from the needles of yew plants. These drugs have a novel 14-member ring, the taxane. Unlike the vinca alkaloids, which cause microtubular disassembly, the taxanes (e.g., taxol) promote microtubular assembly and stability, therefore blocking the cell cycle in mitosis. Other plant derived agents include, but are not limited to, vincristine, vinblastine, vindesine, vinzolidine, vinorelbine, etoposide, teniposide, paclitaxel and docetaxel.

### Immunotherapy Combinations

Other therapeutic anti-cancer treatment regimens include therapeutic immunotherapies such as adoptive cell transfer regimens, antigen-specific vaccination or antibody administration, inhibition of DNA repair proteins (e.g., inhibitors of the nucleic enzyme poly(adenosine 5′-diphospho-ribose) polymerase [“poly(ADP-ribose) polymerase” PARP inhibitors”) and blockade of immune checkpoint inhibitory molecules, for example cytotoxic T lymphocyte-associated antigen 4 (CTLA-4) and programmed death 1 (PD-1) antibodies or their ligands (PDL-1).

Immune checkpoint proteins regulate T cell function in the immune system. T cells play a central role in cell-mediated immunity. Checkpoint proteins interact with specific ligands that send a signal into the T cell and essentially switch off or inhibit T cell function. Cancer cells take advantage of this system by driving high levels of expression of checkpoint proteins on their surface that results in control of the T cells expressing checkpoint proteins on the surface of T cells that enter the tumor microenvironment, thus suppressing the anticancer immune response. As such, inhibition of checkpoint proteins by agents referred to herein as “immune checkpoint protein (ICP) inhibitors” would result in restoration of T cell function and an immune response to the cancer cells. Examples of checkpoint proteins include, but are not limited to: CTLA-4, PDL-1, PDL-2, PD1, B7-H3, B7-H4, BTLA, HVEM, TIM3, GALS, LAG3, VISTA, KIR, 2B4, CD160, CGEN-15049, CHK 1, CHK2, A2aR, OX40, B-7 family ligands or a combination thereof. Preferably, the immune checkpoint inhibitor interacts with a ligand of a checkpoint protein which may be CTLA-4, PDL1, PDL2, PD1, B7-H3, B7-H4, BTLA, HVEM, TIM3, GALS, LAG3, VISTA, KIR, 2B4, CD160, CGEN-15049, CHK 1, CHK2, OX40, A2aR, B-7 family ligands or a combination thereof. Preferably, the checkpoint inhibitor is a biologic therapeutic or a small molecule. Preferably, the checkpoint inhibitor is a monoclonal antibody, a humanized antibody, a fully human antibody, a fusion protein or a combination thereof. Preferably, the PD1 checkpoint inhibitor comprises one or more anti-PD-1 antibodies, including nivolumab and pembrolizumab.

The combination therapy methods described herein include administering at least one checkpoint inhibitor in combination with (S,S)-(HO)2DEHSPM monotherapy or (S,S)-(HO)2DEHSPM/GEM/NAB. The invention is not limited to any specific checkpoint inhibitor so long as the checkpoint inhibitor inhibits one or more activities of the target checkpoint proteins when administered in an effective amount in combination with (S,S)-(HO)2DEHSPM monotherapy or (S,S)-(HO)2DEHSPM/GEM/NAB. In some instances, due to, for example, synergistic effects, minimal inhibition of the checkpoint protein by the checkpoint inhibitor may be sufficient in the presence of (S,S)-(HO)2DEHSPM monotherapy or (S,S)-(HO)2DEHSPM/GEM/NAB. Many checkpoint inhibitors are known in the art, for example, the following is a list of FDA approved checkpoint protein inhibitors:
ipilimumab (YERVOY®)
pembrolizumab (KEYTRUDA®)
atezolizumab (TECENTRIQ®)
durvalumab (IMFINZ®)
avelumab (BAVENCIO®)
nivolumab (OPDIVO®).

A preferred treatment regimen of the invention combines (S,S)-(HO)2DEHSPM monotherapy or (S,S)-(HO)2DEHSPM/GEM/NAB administered in accordance with the invention with the checkpoint inhibitor, pembrolizumab. Preferably, pembrolizumab is administered on the first day of each treatment cycle of the treatment regimen according to the invention. Preferably 200 mg of pembrolizumab is administered in accordance with manufacturer's recommendations, generally once every three weeks or 21 days.

### Antibodies

Preferably the administration of (S,S)-(HO)2DEHSPM monotherapy or (S,S)-(HO)2DEHSPM/GEM/NAB may be combined with a therapeutic antibody. Methods of producing antibodies, and antigen-binding fragments thereof, are well known in the art and are disclosed in, e.g., U.S. Pat. No. 7,247,301, US2008/0138336, and U.S. Pat. No. 7,923,221, all of which are herein incorporated by reference in their entirety. Therapeutic antibodies that can be used in the methods of the present invention include, but are not limited to, any of the art-recognized therapeutic antibodies that are approved for use, in clinical trials, or in development for clinical use. In some embodiments, more than one therapeutic antibody can be included in the combination therapy of the present invention.

Non-limiting examples of therapeutic antibodies include the following, without limitation:
trastuzumab (HERCEPTIN™ by Genentech, South San Francisco, Calif.), which is used to treat HER-2/neu positive breast cancer or metastatic breast cancer;
bevacizumab (AVASTIN™ by Genentech), which is used to treat colorectal cancer, metastatic colorectal cancer, breast cancer, metastatic breast cancer, non-small cell lung cancer, or renal cell carcinoma;
rituximab (RITUXAN™ by Genentech), which is used to treat non-Hodgkin's lymphoma or chronic lymphocytic leukemia;
pertuzumab (OMNITARG™ by Genentech), which is used to treat breast cancer, prostate cancer, non-small cell lung cancer, or ovarian cancer;
cetuximab (ERBITUX™ by ImClone Systems Incorporated, New York, N.Y.), which can be used to treat colorectal cancer, metastatic colorectal cancer, lung cancer, head and neck cancer, colon cancer, breast cancer, prostate cancer, gastric cancer, ovarian cancer, brain cancer, pancreatic cancer, esophageal cancer, renal cell cancer, prostate cancer, cervical cancer, or bladder cancer;
IMC-1C11 (ImClone Systems Incorporated), which is used to treat colorectal cancer, head and neck cancer, as well as other potential cancer targets;
tositumomab and tositumomab and iodine I¹³¹ (BEXXAR™ by Corixa Corporation, Seattle, Wash.), which is used to treat non-Hodgkin's lymphoma, which can be CD20 positive, follicular, non-Hodgkin's lymphoma, with and without transformation, whose disease is refractory to Rituximab and has relapsed following chemotherapy;
In¹¹¹ ibirtumomab tiuxetan; Y⁹⁰ ibirtumomab tiuxetan; ibirtumomab tiuxetan and Y⁹⁰ ibirtumomab tiuxetan (ZEVALIN™ by Biogen Idec, Cambridge, Mass.), which is used to treat lymphoma or non-Hodgkin's lymphoma, which can include relapsed follicular lymphoma; relapsed or refractory, low grade or follicular non-Hodgkin's lymphoma; or transformed B-cell non-Hodgkin's lymphoma;
EMD 7200 (EMD Pharmaceuticals, Durham, N.C.), which is used for treating for treating non-small cell lung cancer or cervical cancer;
SGN-30 (a genetically engineered monoclonal antibody targeted to CD30 antigen by Seattle Genetics, Bothell, Wash.), which is used for treating Hodgkin's lymphoma or non-Hodgkin's lymphoma;
SGN-15 (a genetically engineered monoclonal antibody targeted to a Lewisy-related antigen that is conjugated to doxorubicin by Seattle Genetics), which is used for treating non-small cell lung cancer;
SGN-33 (a humanized antibody targeted to CD33 antigen by Seattle Genetics), which is used for treating acute myeloid leukemia (AML) and myelodysplastic syndromes (MDS);
SGN-40 (a humanized monoclonal antibody targeted to CD40 antigen by Seattle Genetics), which is used for treating multiple myeloma or non-Hodgkin's lymphoma;
SGN-35 (a genetically engineered monoclonal antibody targeted to a CD30 antigen that is conjugated to auristatin E by Seattle Genetics), which is used for treating non-Hodgkin's lymphoma;
SGN-70 (a humanized antibody targeted to CD70 antigen by Seattle Genetics), that is used for treating renal cancer and nasopharyngeal carcinoma;
SGN-75 (a conjugate comprised of the SGN70 antibody and an Auristatin derivative by Seattle Genetics); and
SGN-17/19 (a fusion protein containing antibody and enzyme conjugated to melphalan prodrug by Seattle Genetics), which is used for treating melanoma or metastatic melanoma.

The therapeutic antibodies to be used in the methods of the present invention are not limited to those described herein. For example, the following approved therapeutic antibodies can also be used in the methods of the invention: brentuximab vedotin (ADCETRIS™) for anaplastic large cell lymphoma and Hodgkin lymphoma, ipilimumab (MDX-101; YERVOY™) for melanoma, ofatumumab (ARZERRA™) for chronic lymphocytic leukemia, panitumumab (VECTIBIX™) for colorectal cancer, alemtuzumab (CAMPATH™) for chronic lymphocytic leukemia, ofatumumab (ARZERRA™) for chronic lymphocytic leukemia, gemtuzumab ozogamicin (MYLOTARG™) for acute myelogenous leukemia.

Antibodies for use in accordance with the invention can also target molecules expressed by immune cells, such as, but not limited to, tremelimumab (CP-675,206) and ipilimumab (MDX-010) which targets CTLA4 and has the effect of tumor rejection, protection from re-challenge, and enhanced tumor-specific T cell responses; OX86 which targets OX40 and increases antigen-specific CD8+ T cells at tumor sites and enhances tumor rejection; CT-011 which targets PD 1 and has the effect of maintaining and expanding tumor specific memory T cells and activates NK cells; BMS-663513 which targets CD137 and causes regression of established tumors, as well as the expansion and maintenance of CD8+ T cells, and daclizumab (ZENAPAX™) which targets CD25 and causes transient depletion of CD4+CD25+FOXP3+Tregs and enhances tumor regression and increases the number of effector T cells. A more detailed discussion of these antibodies can be found in, e.g., Weiner et al., Nature Rev. Immunol 2010; 10:317-27.

Preferably, the antibody is a pro-inflammatory and/or pro-tumorigenic cytokine targeting antibody including, but not limited to, anti-TNF antibodies, anti-IL-1Ra receptor targeting antibodies, anti-IL-1 antibodies, anti-IL-6 receptor antibodies, and anti-IL-6 antibodies. Preferably antibodies include those that target pro-inflammatory T helper type 17 cells (TH17).

The therapeutic antibody can be a fragment of an antibody; a complex comprising an antibody; or a conjugate comprising an antibody. The antibody can optionally be chimeric or humanized or fully human.

### Therapeutic Proteins and Polypeptides

Preferably the methods of the invention include administration of the (S,S)-(HO)2DEHSPM monotherapy or (S,S)-(HO)2DEHSPM/GEM/NAB in accordance with the treatment regimen of the invention in combination with a therapeutic protein or peptide. Therapeutic proteins that are effective in treating cancer are well known in the art. Preferably, the therapeutic polypeptide or protein is a “suicide protein” that causes cell death by itself or in the presence of other compounds.

A representative example of such a suicide protein is thymidine kinase of the herpes simplex virus. Additional examples include thymidine kinase of varicella zoster virus, the bacterial gene cytosine deaminase (which converts 5-fluorocytosine to the highly toxic compound 5-fluorouracil), p450 oxidoreductase, carboxypeptidase G2, beta-glucuronidase, penicillin-V-amidase, penicillin-G-amidase, beta-lactamase, nitroreductase, carboxypeptidase A, linamarase (also referred to as (3-glucosidase), the E. coli gpt gene, and the E. coli Deo gene, although others are known in the art. In some embodiments, the suicide protein converts a prodrug into a toxic compound.

As used herein, “prodrug” means any compound useful in the methods of the present invention that can be converted to a toxic product, i.e., toxic to tumor cells. The prodrug is converted to a toxic product by the suicide protein. Representative examples of such prodrugs include: ganciclovir, acyclovir, and FIAU (1-(2-deoxy-2-fluoro-β-D-arabinofuranosyl)-5-iod-ouracil) for thymidine kinase; ifosfamide for oxidoreductase; 6-methoxypurine arabinoside for VZV-TK; 5-fluorocytosine for cytosine deaminase; doxorubicin for beta-glucuronidase; CB 1954 and nitrofurazone for nitroreductase; and N-(Cyanoacetyl)-L-phenylalanine or N-(3-chloropropionyl)-L-phenylalanine for carboxypeptidase A. The prodrug may be administered readily by a person having ordinary skill in this art. A person with ordinary skill would readily be able to determine the most appropriate dose and route for the administration of the prodrug.

Preferably the therapeutic protein or polypeptide, is a cancer suppressor, for example p53 or Rb, or a nude acid encoding such a protein or polypeptide. Those of skill know of a wide variety of such cancer suppressors and how to obtain them and/or the nucleic acids encoding them.

Other examples of anti-cancer/therapeutic proteins or polypeptides include pro-apoptotic therapeutic proteins and polypeptides, for example, p15, p16, or p21^{WAF-1}.

Cytokines, and nucleic acid encoding them may also be used as therapeutic proteins and polypeptides. Examples include: GM-CSF (granulocyte macrophage colony stimulating factor); TNF-alpha (Tumor necrosis factor alpha); Interferons including, but not limited to, IFN-alpha and IFN-gamma; and Interleukins including, but not limited to, Interleukin-1 (IL-1), Interleukin-Beta (IL-beta), Interleukin-2 (IL-2), Interleukin-4 (IL-4), Interleukin-5 (IL-5), Interleukin-6 (IL-6), Interleukin-7 (IL-7), Interleukin-8 (IL-8), Interleukin-10 (IL-10), Interleukin-12 (IL-12), Interleukin-13 (IL-13), Interleukin-14 (IL-14), Interleukin-15 (IL-15), Interleukin-16 (IL-16), Interleukin-18 (IL-18), Interleukin-23 (IL-23), Interleukin-24 (IL-24), although other embodiments are known in the art.

Additional examples of cytocidal genes includes, but is not limited to, mutated cyclin G1 genes. By way of example, the cytocidal gene may be a dominant negative mutation of the cyclin G1 protein (e.g., WO/01/64870).

### Vaccines

Preferably, the therapeutic regimens of the invention include administration of (S,S)-(HO)2DEHSPM monotherapy or (S,S)-(HO)2DEHSPM/GEM/NAB in combination with administration of a cancer vaccine for stimulating a cancer specific-immune response, e.g., innate and adaptive immune responses, for generating host immunity against a cancer. Illustrative vaccines include, but are not limited to, for example, antigen vaccines, whole cell vaccines, dendritic cell vaccines, and DNA vaccines. Depending upon the particular type of vaccine, the vaccine composition may include one or more suitable adjuvants known to enhance a subject's immune response to the vaccine.

The vaccine may, for example, be cellular based, i.e., created using cells from the patient's own cancer cells to identify and obtain an antigen. Exemplary vaccines include tumor cell-based and dendritic-cell based vaccines, where activated immune cells from the subject are delivered back to the same subject, along with other proteins, to further facilitate immune activation of these tumor antigen primed immune cells. Tumor cell-based vaccines include whole tumor cells and gene-modified tumor cells. Whole tumor cell vaccines may optionally be processed to enhance antigen presentation, e.g., by irradiation of either the tumor cells or tumor lysates). Vaccine administration may also be accompanied by adjuvants such as bacillus calmette-guerin (BCG) or keyhole limpet hemocyanin (KLH), depending upon the type of vaccine employed. Plasmid DNA vaccines may also be used and can be administered via direct injection or biolistically. Also contemplated for use are peptide vaccines, viral gene transfer vector vaccines, and antigen-modified dentritic cells (DCs).

Preferably the vaccine is a therapeutic cancer peptide-based vaccine. Peptide vaccines can be created using known sequences or from isolated antigens from a subject's own tumor(s) and include neoantigens and modified antigens. Illustrative antigen-based vaccines include those where the antigen is a tumor-specific antigen. For example, the tumor-specific antigen may be selected from a cancer-testis antigen, a differentiation antigen, and a widely occurring over-expressed tumor associated antigen, among others. Recombinant peptide vaccines, based on peptides from tumor-associated antigens, when used in the instant method, may be administered or formulated with, an adjuvant or immune modulator. Illustrative antigens for use in a peptide-based vaccine include, but are not limited to, the following, since this list is meant to be purely illustrative. For example, a peptide vaccine may comprise a cancer-testis antigen such as MAGE, BAGE, NY-ESO-1 and SSX-2, encoded by genes that are normally silenced in adult tissues but transcriptionally reactivated in tumor cells. Alternatively, the peptide vaccine may comprise a tissue differentiation associated antigen, i.e., an antigen of normal tissue origin and shared by both normal and tumorous tissue. For example, the vaccine may comprise a melanoma-associated antigen such as gp100, Melan-A/Mart-1, MAGE-3, or tyrosinase; or may comprise a prostate cancer antigen such as PSA or PAP. The vaccine may comprise a breast cancer-associated antigen such as mammaglobin-A. Other tumor antigens that may be comprised in a vaccine for use in the instant method include, for example, CEA, MUC-1, HER1/Nue, hTERT, ras, and B-raf. Other suitable antigens that may be used in a vaccine include SOX-2 and OCT-4, associated with cancer stem cells or the epithelial-to-mesenchymal transition process.

Antigen vaccines include multi-antigen and single antigen vaccines. Exemplary cancer antigens may include peptides having from about 5 to about 30 amino acids, or from about 6 to 25 amino acids, or from about 8 to 20 amino acids.

As described above, an immunostimulatory adjuvant (different from RSLAIL-2) may be used in a vaccine, in particular, a tumor-associated antigen-based vaccine, to assist in generating an effective immune response. For example, a vaccine may incorporate a pathogen-associated molecular pattern (PAMP) to assist in improving immunity. Additional suitable adjuvants include monophosphoryl lipid A, or other lipopolysaccharides; toll-like receptor (TLR) agonists such as, for example, imiquimod, resiquimod (R-848), TLR3, IMO-8400, and rintatolimod. Additional adjuvants suitable for use include heat shock proteins or inhibitors of heat shock proteins.

A genetic vaccine typically uses viral or plasmid DNA vectors carrying expression cassettes. Upon administration, they transfect somatic cells or dendritic cells as part of the inflammatory response to thereby result in cross-priming or direct antigen presentation. Preferably, a genetic vaccine is one that provides delivery of multiple antigens in one immunization. Genetic vaccines include DNA vaccines, RNA vaccines and viral-based vaccines.

DNA vaccines for use in the instant methods are bacterial plasmids that are constructed to deliver and express tumor antigen. DNA vaccines may be administered by any suitable mode of administration, e.g., subcutaneous or intradermal injection, but may also be injected directly into the lymph nodes. Additional modes of delivery include, for example, gene gun, electroporation, ultrasound, laser, liposomes, microparticles and nanoparticles.

Preferably, the vaccine comprises a neoantigen, or multiple neoantigens. Preferably, the vaccine is a neoantigen-based vaccine. Preferably a neoantigen-based vaccine (NBV) composition may encode multiple cancer neoantigens in tandem, where each neoantigen is a polypeptide fragment derived from a protein mutated in cancer cells. For instance, a neoantigenic vaccine may comprise a first vector comprising a nucleic acid construct encoding multiple immunogenic polypeptide fragments, each of a protein mutated in cancer cells, where each immunogenic polypeptide fragment comprises one or more mutated amino acids flanked by a variable number of wild type amino acids from the original protein, and each polypeptide fragment is joined head-to-tail to form an immunogenic polypeptide. The lengths of each of the immunogenic polypeptide fragments forming the immunogenic polypeptide can vary.

Viral gene transfer vector vaccines may also be used; in such vaccines, recombinant engineered virus, yeast, bacteria or the like is used to introduce cancer-specific proteins to the patient's immune cells. In a vector-based approach, which can be tumor lytic or non-tumor lytic, the vector can increase the efficiency of the vaccine due to, for example, its inherent immunostimulatory properties. Illustrative viral-based vectors include those from the poxviridae family, such as vaccinia, modified vaccinia strain Ankara and avipoxviruses. Also suitable for use is the cancer vaccine, PROSTVAC, containing a replication-competent vaccinia priming vector and a replication-incompetent fowlbox-boosting vector. Each vector contains transgenes for PSA and three co-stimulatory molecules, CD80, CD54 and CD58, collectively referred to as TRICOM. Other suitable vector-based cancer vaccines include Trovax and TG4010 (encoding MUC1 antigen and IL-2). Additional vaccines for use include bacteria and yeast-based vaccines such as recombinant Listeria monocytogenes and Saccharomyces cerevisae.

The foregoing vaccines may be combined and/or formulated with adjuvants and other immune boosters to increase efficacy. Depending upon the particular vaccine, administration may be either intratumoral or non-intratumoral (i.e., systemic).

### Small Molecules

Preferably, the therapeutic regimens of the invention include administration of (S,S)-(HO)2DEHSPM monotherapy or (S,S)-(HO)2DEHSPM/GEM/NAB in combination with administration of an anticancer small molecule. Small molecules that are effective in treating cancer are well known in the art and include antagonists of factors that are involved in tumor growth, such as EGFR, ErbB2 (also known as Her2/neu) ErbB3, ErbB4, or TNF. Non-limiting examples include small molecule receptor tyrosine kinase inhibitors (RTKIs) that target one or more tyrosine kinase receptors, such as VEGF receptors, FGF receptors, EGF receptors and PDGF receptors.

Many therapeutic small molecule RTKIs are known in the art, including, but are not limited to, vatalanib (PTK787), erlotinib (TARCEVA™), OSI-7904, ZD6474 (ZACTIMA™), ZD6126 (ANG453), ZD1839, sunitinib (SUTENT™), semaxanib (SU5416), AMG706, AG013736, Imatinib (GLEEVEC™), MLN-518, CEP-701, PKC-412, Lapatinib (GSK572016), VELCADE™, AZD2171, sorafenib (NEXAVAR™), XL880, and CHIR-265. Small molecule protein tyrosine phosphatase inhibitors, such as those disclosed in Jiang et al., Cancer Metastasis Rev. 2008; 27:263-72 are also useful for practicing the methods of the invention. Such inhibitors can target, e.g., HSP2, PRL, PTP1B, or Cdc25 phosphatases.

Small molecules that target Bcl-2/Bcl-XL, such as those disclosed in US2008/0058322, are also useful for practicing the methods of the present invention. Further exemplary small molecules for use in the present invention are disclosed in Zhang et al. Nature Reviews: Cancer 2009; 9:28-39. In particular, chemotherapeutic agents that lead to immunogenic cell death such as anthracyclins (Kepp et al., Cancer and Metastasis Reviews 2011; 30:61-9) will be well suited for synergistic effects with extended-PK IL-2.

### Cancer Antigens

Preferably, the methods of the invention include administration of the (S,S)-(HO)2DEHSPM monotherapy or (S,S)-(HO)2DEHSPM/GEM/NAB in combination with administration of a cancer antigen, e.g., for use as a cancer vaccine (see, e.g., Overwijk, et al. Journal of Experimental Medicine 2008; 198:569-80). Other cancer antigens that can be used in vaccinations include, but are not limited to, (i) tumor-specific antigens, (ii) tumor-associated antigens, (iii) cells that express tumor-specific antigens, (iv) cells that express tumor-associated antigens, (v) embryonic antigens on tumors, (vi) autologous tumor cells, (vii) tumor-specific membrane antigens, (viii) tumor-associated membrane antigens, (ix) growth factor receptors, (x) growth factor ligands, and (xi) any other type of antigen or antigen-presenting cell or material that is associated with a cancer.

The cancer antigen may be an epithelial cancer antigen, (e.g., breast, gastrointestinal, lung), a prostate specific cancer antigen (PSA) or prostate specific membrane antigen (PSMA), a bladder cancer antigen, a lung (e.g., small cell lung) cancer antigen, a colon cancer antigen, an ovarian cancer antigen, a brain cancer antigen, a gastric cancer antigen, a renal cell carcinoma antigen, a pancreatic cancer antigen, a liver cancer antigen, an esophageal cancer antigen, a head and neck cancer antigen, or a colorectal cancer antigen.

In another embodiment, the cancer antigen is a lymphoma antigen (e.g., non-Hodgkin's lymphoma or Hodgkin's lymphoma), a B-cell lymphoma cancer antigen, a leukemia antigen, a myeloma (i.e., multiple myeloma or plasma cell myeloma) antigen, an acute lymphoblastic leukemia antigen, a chronic myeloid leukemia antigen, or an acute myelogenous leukemia antigen. The described cancer antigens are only exemplary, and that any cancer antigen can be targeted in the present invention.

Preferably, the cancer antigen is a mucin-1 protein or peptide (MUC-1) that is found on all human adenocarcinomas: pancreas, colon, breast, ovarian, lung, prostate, head and neck, including multiple myelomas and some B cell lymphomas. Patients with inflammatory bowel disease, either Crohn's disease or ulcerative colitis, are at an increased risk for developing colorectal carcinoma. MUC-1 is a type I transmembrane glycoprotein. The major extracellular portion of MUC-1 has a large number of tandem repeats consisting of 20 amino acids which comprise immunogenic epitopes. In some cancers it is exposed in an unglycosylated form that is recognized by the immune system (Gendler et al., J Biol Chem 1990; 265:15286-15293).

In another embodiment, the cancer antigen is a mutated B-Raf antigen, which is associated with melanoma and colon cancer. The vast majority of these mutations represent a single nucleotide change of T-A at nucleotide 1796 resulting in a valine to glutamic acid change at residue 599 within the activation segment of B-Raf. Raf proteins are also indirectly associated with cancer as effectors of activated Ras proteins, oncogenic forms of which are present in approximately one-third of all human cancers. Normal non-mutated B-Raf is involved in cell signaling, relaying signals from the cell membrane to the nucleus. The protein is usually only active when needed to relay signals. In contrast, mutant B-Raf has been reported to be constantly active, disrupting the signaling relay (Mercer and Pritchard, Biochim Biophys Acta (2003) 1653(1):25-40; Sharkey et al., Cancer Res. (2004) 64(5):1595-1599).

Preferably, the cancer antigen is a human epidermal growth factor receptor-2 (HER-2/neu) antigen. Cancers that have cells that overexpress HER-2/neu are referred to as HER-2/neu⁺ cancers. Exemplary HER-2/neu⁺ cancers include prostate cancer, lung cancer, breast cancer, ovarian cancer, pancreatic cancer, skin cancer, liver cancer (e.g., hepatocellular adenocarcinoma), intestinal cancer, and bladder cancer.

HER-2/neu has an extracellular binding domain (ECD) of approximately 645 aa, with 40% homology to epidermal growth factor receptor (EGFR), a highly hydrophobic transmembrane anchor domain (TMD), and a carboxyterminal intracellular domain (ICD) of approximately 580 aa with 80% homology to EGFR. The nucleotide sequence of HER-2/neu is available at GENBANK™. Accession Nos. AH002823 (human HER-2 gene, promoter region and exon 1); M16792 (human HER-2 gene, exon 4): M16791 (human HER-2 gene, exon 3); M16790 (human HER-2 gene, exon 2); and M16789 (human HER-2 gene, promoter region and exon 1). The amino acid sequence for the HER-2/neu protein is available at GENBANK™ Accession No. AAA58637. Based on these sequences, one skilled in the art could develop HER-2/neu antigens using known assays to find appropriate epitopes that generate an effective immune response.

Exemplary HER-2/neu antigens include p369-377 (a HER-2/neu derived HLA-A2 peptide); dHER2 (Corixa Corporation); li-Key MHC class II epitope hybrid (Generex Biotechnology Corporation); peptide P4 (amino acids 378-398); peptide P7 (amino acids 610-623); mixture of peptides P6 (amino acids 544-560) and P7; mixture of peptides P4, P6 and P7; HER2 [9754]; and the like.

Preferably, the cancer antigen is an epidermal growth factor receptor (EGFR) antigen. The EGFR antigen can be an EGFR variant 1 antigen, an EGFR variant 2 antigen, an EGFR variant 3 antigen and/or an EGFR variant 4 antigen. Cancers with cells that overexpress EGFR are referred to as EGFR cancers. Exemplary EGFR cancers include lung cancer, head and neck cancer, colon cancer, colorectal cancer, breast cancer, prostate cancer, gastric cancer, ovarian cancer, brain cancer and bladder cancer.

Preferably, the cancer antigen is a vascular endothelial growth factor receptor (VEGFR) antigen. VEGFR is considered to be a regulator of cancer-induced angiogenesis. Cancers with cells that overexpress VEGFR are called VEGFR⁺ cancers. Exemplary VEGFR⁺ cancers include breast cancer, lung cancer, small cell lung cancer, colon cancer, colorectal cancer, renal cancer, leukemia, and lymphocytic leukemia.

Preferably, the cancer antigen is prostate-specific antigen (PSA) and/or prostate-specific membrane antigen (PSMA) that are prevalently expressed in androgen-independent prostate cancers.

Preferably, the cancer antigen is Gp-100 Glycoprotein 100 (gp 100) is a tumor-specific antigen associated with melanoma.

Preferably, the cancer antigen is a carcinoembryonic (CEA) antigen. Cancers with cells that overexpress CEA are referred to as CEA⁺ cancers. Exemplary CEA⁺ cancers include colorectal cancer, gastric cancer and pancreatic cancer. Exemplary CEA antigens include CAP-1 (i.e., CEA aa 571-579), CAP1-6D, CAP-2 (i.e., CEA aa 555-579), CAP-3 (i.e., CEA aa 87-89), CAP-4 (CEA aa 1-11), CAP-5 (i.e., CEA aa 345-354), CAP-6 (i.e., CEA aa 19-28) and CAP-7.

Preferably, the cancer antigen is carbohydrate antigen 19-9 (CA 19-9). CA 19-9 is an oligosaccharide related to the Lewis A blood group substance and is associated with colorectal cancers.

Preferably, the cancer antigen is a melanoma cancer antigen. Melanoma cancer antigens are useful for treating melanoma. Exemplary melanoma cancer antigens include MART-1 (e.g., MART-1 26-35 peptide, MART-1 27-35 peptide); MART-1/Melan A; pMel17; pMel17/gp100; gp100 (e.g., gp 100 peptide 280-288, gp 100 peptide 154-162, gp 100 peptide 457-467); TRP-1; TRP-2; NY-ESO-1; p16; beta-catenin; mum-1; and the like.

Preferably, the cancer antigen is a mutant or wild type ras peptide. The mutant ras peptide can be a mutant K-ras peptide, a mutant N-ras peptide and/or a mutant H-ras peptide. Mutations in the ras protein typically occur at positions 12 (e.g., arginine or valine substituted for glycine), 13 (e.g., asparagine for glycine), 61 (e.g., glutamine to leucine) and/or 59. Mutant ras peptides can be useful as lung cancer antigens, gastrointestinal cancer antigens, hepatoma antigens, myeloid cancer antigens (e.g., acute leukemia, myelodysplasia), skin cancer antigens (e.g., melanoma, basal cell, squamous cell), bladder cancer antigens, colon cancer antigens, colorectal cancer antigens, and renal cell cancer antigens.

In another embodiment of the invention, the cancer antigen is a mutant and/or wildtype p53 peptide. The p53 peptide can be used as colon cancer antigens, lung cancer antigens, breast cancer antigens, hepatocellular carcinoma cancer antigens, lymphoma cancer antigens, prostate cancer antigens, thyroid cancer antigens, bladder cancer antigens, pancreatic cancer antigens and ovarian cancer antigens.

The cancer antigen can be a cell, a protein, a peptide, a fusion protein, DNA encoding a peptide or protein, RNA encoding a peptide or protein, a glycoprotein, a lipoprotein, a phosphoprotein, a carbohydrate, a lipopolysaccharide, a lipid, a chemically linked combination of two or more thereof, a fusion or two or more thereof, or a mixture of two or more thereof, or a virus encoding two or more thereof, or an oncolytic virus encoding two or more thereof. In another embodiment, the cancer antigen is a peptide comprising about 6 to about 24 amino acids; from about 8 to about 20 amino acids; from about 8 to about 12 amino acids; from about 8 to about 10 amino acids; or from about 12 to about 20 amino acids. In one embodiment, the cancer antigen is a peptide having a MHC Class I binding motif or a MHC Class II binding motif. In another embodiment, the cancer antigen comprises a peptide that corresponds to one or more cytotoxic T lymphocyte (CTL) epitopes.

### Cell Therapy

Preferably, the methods of the invention include administration of (S,S)-(HO)2DEHSPM monotherapy or (S,S)-(HO)2DEHSPM/GEM/NAB in combination with administration of a therapeutic cell therapy. Cell therapies that are useful for treating cancer are well known and are disclosed in, e.g., U.S. Pat. No. 7,402,431. In a preferred embodiment, the cell therapy is T cell transplant. In a preferred method, T cells are expanded ex vivo with IL-2 prior to transplantation into a subject. Methods for cell therapies are disclosed in, e.g., U.S. Pat. No. 7,402,431, US2006/0057121, U.S. Pat. Nos. 5,126,132, 6,255,073, 5,846,827, 6,251,385, 6,194,207, 5,443,983, 6,040,177, 5,766,920, and US2008/0279836.

### Cancer Indications

(S,S)-(HO)2DEHSPM is a dysfunctional analogue of the naturally occurring polyamine spermine. It inhibits cell growth by substituting for spermine, reduces spermine levels and depletes intracellular pools of spermidine and putrescine. The polyamine transport uptake mechanism appears to be up-regulated in various tumor types. This increase in biosynthesis of polyamines and their biosynthetic enzymes in neoplastic tissues can be leveraged to target (S,S)-(HO)2DEHSPM to tumor cells, particularly solid tumor cells resulting in tumor growth inhibition and cell death.

A tumor can be classified as malignant or benign. In both cases, there is an abnormal aggregation and proliferation of cells. In the case of a malignant tumor, these cells behave more aggressively, acquiring properties of increased invasiveness. Ultimately, the tumor cells may even gain the ability to break away from the microscopic environment in which they originated, spread to another area of the body (with a very different environment, not normally conducive to their growth), and continue their rapid growth and division in this new location. This is called metastasis. Once malignant cells have metastasized, achieving a cure is more difficult. Benign tumors do not invade or metastasize.

Inhibition or reduction of tumor growth refers to a reduction in the size or volume of a tumor mass, a decrease in the number and/or size of metastasized tumors in a subject, a decrease in the proliferative status (the degree to which the cancer cells are multiplying) of the cancer cells, and the like.

The treatment regimens of the invention are particularly suited for treating solid tumors including but not limited to: pancreatic adenocarcinoma (e.g., PDA), colorectal adenocarcinoma, prostate adenocarcinoma, breast carcinoma, lung adenocarcinoma, cholangiocarcinoma, lymphomas, melanoma, renal cell carcinoma (RCC), hepatic cell carcinoma (HCC), ovarian cell tumors and including advanced solid tumors and tumors that have previously been treated with anti-cancer therapy but remain refractory to previous therapies.

Given the upregulation of polyamine synthesis in solid tumors and other types of cancer, (S,S)-(HO)2DEHSPM monotherapy or (S,S)-(HO)2DEHSPM/GEM/NAB treatment regimens of the invention are useful in the treatment of many types of cancer. The term “cancer”, as used herein, shall be given its ordinary meaning, as a general term for diseases in which abnormal cells divide with attenuated control.

Cancer cells can invade nearby tissues and can spread through the bloodstream and lymphatic system to other parts of the body. There are several main types of cancer, for example, carcinoma is cancer that begins in the skin or in tissues that line or cover internal organs and is derived from the epithelium. Sarcoma is cancer that begins in bone, cartilage, fat, muscle, blood vessels, or other connective or supportive tissue derived from mesothelium. Leukemia is cancer that starts in blood-forming tissue such as the bone marrow and causes large numbers of abnormal blood cells to be produced and enter the bloodstream. Lymphoma is cancer that begins in the cells of the non-hematogenous immune system.

When normal cells lose their ability to behave as a specified, controlled and coordinated unit, a tumor is formed. Generally, a solid tumor is an abnormal mass of tissue that usually does not contain cysts or liquid areas (some brain tumors do have cysts and central necrotic areas filled with liquid). A single tumor may even have different populations of cells within it, with differing processes that have gone awry. Solid tumors may be benign (not cancerous), or malignant (cancerous). Different types of solid tumors are named for the type of cells that form them. Examples of solid tumors are sarcomas, carcinomas, and lymphomas. Leukemias (cancers of the blood) generally do not form solid tumors.

Representative cancers include, but are not limited to, Acute Lymphoblastic Leukemia, Adult; Acute Lymphoblastic Leukemia, Childhood; Acute Myeloid Leukemia, Adult; Adrenocortical Carcinoma; Adrenocortical Carcinoma, Childhood; AIDS-Related Lymphoma; AIDS-Related Malignancies; Anal Cancer; Astrocytoma, Childhood Cerebellar; Astrocytoma, Childhood Cerebral; Bile Duct Cancer, Extrahepatic; Bladder Cancer; Bladder Cancer, Childhood; Bone Cancer, Osteosarcoma/Malignant Fibrous Histiocytoma; Glioblastoma, Childhood; Glioblastoma, Adult; Brain Stem Glioma, Childhood; Brain Tumor, Adult; Brain Tumor, Brain Stem Glioma, Childhood; Brain Tumor, Cerebellar Astrocytoma, Childhood; Brain Tumor, Cerebral Astrocytoma/Malignant Glioma, Childhood; Brain Tumor, Ependymoma, Childhood; Brain Tumor, Medulloblastoma, Childhood; Brain Tumor, Supratentorial Primitive Neuroectodermal Tumors, Childhood; Brain Tumor, Visual Pathway and Hypothalamic Glioma, Childhood; Brain Tumor, Childhood (Other); Breast Cancer; Breast Cancer and Pregnancy; Breast Cancer, Childhood; Breast Cancer, Male; Bronchial Adenomas/Carcinoids, Childhood: Carcinoid Tumor, Childhood; Carcinoid Tumor, Gastrointestinal; Carcinoma, Adrenocortical; Carcinoma, Islet Cell; Carcinoma of Unknown Primary; Central Nervous System Lymphoma, Primary; Cerebellar Astrocytoma, Childhood; Cerebral Astrocytoma/Malignant Glioma, Childhood; Cervical Cancer; Childhood Cancers; Chronic Lymphocytic Leukemia; Chronic Myelogenous Leukemia; Chronic Myeloproliferative Disorders; Clear Cell Sarcoma of Tendon Sheaths; Colon Cancer; Colorectal Cancer, Childhood; Cutaneous T-Cell Lymphoma; Endometrial Cancer; Ependymoma, Childhood; Epithelial Cancer, Ovarian; Esophageal Cancer; Esophageal Cancer, Childhood; Ewing's Family of Tumors; Extracranial Germ Cell Tumor, Childhood; Extragonadal Germ Cell Tumor; Extrahepatic Bile Duct Cancer; Eye Cancer, Intraocular Melanoma; Eye Cancer, Retinoblastoma; Gallbladder Cancer; Gastric (Stomach) Cancer; Gastric (Stomach) Cancer, Childhood; Gastrointestinal Carcinoid Tumor; Germ Cell Tumor, Extracranial, Childhood; Germ Cell Tumor, Extragonadal; Germ Cell Tumor, Ovarian; Gestational Trophoblastic Tumor; Glioma. Childhood Brain Stem; Glioma. Childhood Visual Pathway and Hypothalamic; Hairy Cell Leukemia; Head and Neck Cancer; Hepatocellular (Liver) Cancer, Adult (Primary); Hepatocellular (Liver) Cancer, Childhood (Primary); Hodgkin's Lymphoma, Adult; Hodgkin's Lymphoma, Childhood; Hodgkin's Lymphoma During Pregnancy; Hypopharyngeal Cancer; Hypothalamic and Visual Pathway Glioma, Childhood; Intraocular Melanoma; Islet Cell Carcinoma (Endocrine Pancreas); Kaposi's Sarcoma; Kidney Cancer; Laryngeal Cancer; Laryngeal Cancer, Childhood; Leukemia, Acute Lymphoblastic, Adult; Leukemia, Acute Lymphoblastic, Childhood; Leukemia, Acute Myeloid, Adult; Leukemia, Acute Myeloid, Childhood; Leukemia, Chronic Lymphocytic; Leukemia, Chronic Myelogenous; Leukemia, Hairy Cell; Lip and Oral Cavity Cancer; Liver Cancer, Adult (Primary); Liver Cancer, Childhood (Primary); Lung Cancer, Non-Small Cell Lung Cancer, Small Cell Lung Cancer; Lymphoblastic Leukemia, Adult Acute; Lymphoblastic Leukemia, Childhood Acute; Lymphocytic Leukemia, Chronic; Lymphoma, AIDS-Related; Lymphoma, Central Nervous System (Primary); Lymphoma, Cutaneous T-Cell; Lymphoma, Hodgkin's, Adult; Lymphoma, Hodgkin's; Childhood; Lymphoma, Hodgkin's During Pregnancy; Lymphoma, Non-Hodgkin's, Adult; Lymphoma, Non-Hodgkin's, Childhood; Lymphoma, Non-Hodgkin's During Pregnancy; Lymphoma, Primary Central Nervous System; Macroglobulinemia, Waldenstrom's; Male Breast Cancer; Malignant Mesothelioma, Adult; Malignant Mesothelioma, Childhood; Malignant Thymoma; Medulloblastoma, Childhood; Melanoma; Melanoma, Intraocular; Merkel Cell Carcinoma; Mesothelioma, Malignant; Metastatic Squamous Neck Cancer with Occult Primary; Multiple Endocrine Neoplasia Syndrome, Childhood; Multiple Myeloma/Plasma Cell Neoplasm; Mycosis Fungoides; Myelodysplastic Syndromes; Myelogenous Leukemia, Chronic; Myeloid Leukemia, Childhood Acute; Myeloma, Multiple; Myeloproliferative Disorders, Chronic; Nasal Cavity and Paranasal Sinus Cancer; Nasopharyngeal Cancer; Nasopharyngeal Cancer, Childhood; Neuroblastoma; Neurofibroma; Non-Hodgkin's Lymphoma, Adult; Non-Hodgkin's Lymphoma, Childhood; Non-Hodgkin's Lymphoma During Pregnancy; Non-Small Cell Lung Cancer; Oral Cancer, Childhood; Oral Cavity and Lip Cancer; Oropharyngeal Cancer; Osteosarcoma/Malignant Fibrous Histiocytoma of Bone; Ovarian Cancer, Childhood; Ovarian Epithelial Cancer; Ovarian Germ Cell Tumor; Ovarian Low Malignant Potential Tumor; Pancreatic Cancer; Childhood, Pancreatic Cancer, Islet Cell; Paranasal Sinus and Nasal Cavity Cancer; Parathyroid Cancer; Penile Cancer; Pheochromocytoma; Pineal and Supratentorial Primitive Neuroectodermal Tumors, Childhood; Pituitary Tumor; Plasma Cell Neoplasm/Multiple Myeloma; Pleuropulmonary Blastoma; Pregnancy and Breast Cancer; Pregnancy and Hodgkin's Lymphoma; Pregnancy and Non-Hodgkin's Lymphoma; Primary Central Nervous System Lymphoma; Primary Liver Cancer, Adult; Primary Liver Cancer, Childhood; Prostate Cancer; Rectal Cancer; Renal Cell (Kidney) Cancer; Renal Cell Cancer, Childhood; Renal Pelvis and Ureter, Transitional Cell Cancer; Retinoblastoma; Rhabdomyosarcoma, Childhood; Salivary Gland Cancer; Salivary Gland’ Cancer, Childhood; Sarcoma, Ewing's Family of Tumors; Sarcoma, Kaposi's; Sarcoma (Osteosarcoma)/Malignant Fibrous Histiocytoma of Bone; Sarcoma, Rhabdomyosarcoma, Childhood; Sarcoma, Soft Tissue, Adult; Sarcoma, Soft Tissue, Childhood; Sezary Syndrome; Skin Cancer; Skin Cancer, Childhood; Skin Cancer (Melanoma); Skin Carcinoma, Merkel Cell; Small Cell Lung Cancer; Small Intestine Cancer; Soft Tissue Sarcoma, Adult; Soft Tissue Sarcoma, Childhood; Squamous Neck Cancer with Occult Primary, Metastatic; Stomach (Gastric) Cancer; Stomach (Gastric) Cancer, Childhood; Supratentorial Primitive Neuroectodermal Tumors, Childhood; T-Cell Lymphoma, Cutaneous; Testicular Cancer; Thymoma, Childhood; Thymoma, Malignant; Thyroid Cancer; Thyroid Cancer, Childhood; Transitional Cell Cancer of the Renal Pelvis and Ureter; Trophoblastic Tumor, Gestational; Unknown Primary Site, Cancer of, Childhood; Unusual Cancers of Childhood; Ureter and Renal Pelvis, Transitional Cell Cancer; Urethral Cancer; Uterine Sarcoma; Vaginal Cancer; Visual Pathway and Hypothalamic Glioma, Childhood; Vulvar Cancer; Waldenstrom's Macro globulinemia; and Wilms' Tumor, among others.

### Kits

Also provided are kits comprising (S,S)-(HO)2DEHSPM formulated for administration by injection, and optionally any other chemotherapeutic or anti-cancer agent including, but not limited to GEM and NAB. The kits are generally in the form of a physical structure housing various components, as described below, and can be utilized, for example, in practicing the methods described above. A kit can include (S,S)-(HO)2DEHSPM (provided in, e.g., a sterile container), which can be in the form of a pharmaceutical composition suitable for administration to a subject, for example, in a prefilled syringe. The pharmaceutical composition can be provided in a form that is ready for use or in a form requiring, for example, reconstitution or dilution prior to administration. When they compositions are in a form that needs to be reconstituted by a user, the kit can also include buffers, pharmaceutically acceptable excipients, and the like, packaged with or separately from (S,S)-(HO)2DEHSPM. When combination therapy is contemplated, the kit can contain the several agents separately or they can already be combined in the kit. Similarly, when additional complementary therapy is required (e.g., (S,S)-(HO)2DEHSPM monotherapy or (S,S)-(HO)2DEHSPM/GEMNAB combination therapy in further combination an additional complementary therapy or agent), the kit can contain the several agents separately or two or more of them can already be combined in the kit.

A kit of the invention can be designed for conditions necessary to properly maintain the components housed therein (e.g., refrigeration or freezing). A kit can contain a label or packaging insert including identifying information for the components therein and instructions for their use (e.g., dosing parameters, clinical pharmacology of the active ingredient(s), including mechanism(s) of action, pharmacokinetics and pharmacodynamics, adverse effects, contraindications, etc.).

Each component of the kit can be enclosed within an individual container, and all of the various containers can be within a single package. Labels or inserts can include manufacturer information such as lot numbers and expiration dates. The label or packaging insert can be, e.g., integrated into the physical structure housing the components, contained separately within the physical structure, or affixed to a component of the kit (e.g., an ampule, syringe or vial).

Labels or inserts can additionally include, or be incorporated into, a computer readable medium, such as a disk (e.g., hard disk, card, memory disk), optical disk such as CD- or DVD-ROM/RAM, DVD, MP3, magnetic tape, or an electrical storage media such as RAM and ROM or hybrids of these such as magnetic/optical storage media, FLASH media or memory-type cards. In some embodiments, the actual instructions are not present in the kit, but means for obtaining the instructions from a remote source, e.g., via an internet site, are provided.

The following examples are offered by way of illustration and are not to be construed as limiting the invention as claimed in any way.

### EXAMPLES

### Example 1: Efficacy of (S,S)-(HO)2DEHSPM Against Human Pancreatic Ductal Adenocarcinoma Following Orthotopic Implantation of L3.6pl Pancreatic Cancer Cells into Nude Mice

Background:

(S,S)-(HO)2DEHSPM is an analogue of the native polyamine (PA), spermine. Increases in polyamine biosynthesis, which occur in a number of neoplasms, including pancreatic ductal adenocarcinoma (PDA), suggest this to be a promising therapeutic target. These studies examined the anti-neoplastic effects of subcutaneous administration of (S,S)-(HO)2DEHSPM following orthotopic implantation of human L3.6pl pancreatic cancer cells into the pancreas of nude mice.

In this and subsequent examples, (S,S)-(HO)2DEHSPM is dosed or administered as the tetrahydrochloride salt, (S,S)-(HO)2DEHSPM.4HCl, and the doses or amounts disclosed refer to the mass of this salt.

### Methods:

L3.6pl cells were injected into the pancreas of nude mice; treatment was initiated by group (n=10/group planned) 7-10 days later following establishment of tumors:

Study 1: saline (control), (S,S)-(HO)2DEHSPM (25 mg/kg), (S,S)-(HO)2DEHSPM (50 mg/kg), and (S,S)-(HO)2DEHSPM (100 mg/kg) daily (QD) for 4 to 6 weeks (wks).

Study 2: saline (control), (S,S)-(HO)2DEHSPM (25 mg/kg QD), (S,S)-(HO)2DEHSPM (25 mg/kg 3×/wk), (S,S)-(HO)2DEHSPM (15 mg/kg 3×/wk), and (S,S)-(HO)2DEHSPM (5 mg/kg 3×/wk) for 4 to 6 wks.

Study 3: saline (control), gemcitabine (GEM) (100 mg/kg 2×/wk, intraperitoneally), (S,S)-(HO)2DEHSPM (25 mg/kg 3×/wk), and (S,S)-(HO)2DEHSPM+GEM for 4 to 6 wks.

### Results:

In Study 1, the 25 mg/kg QD dosing regimen resulted in an 82.9% reduction in pancreas weight in tumor bearing mice. Doses of 50 and 100 mg/kg resulted in earlier deaths and proved to be toxic. Histologic changes in the liver included hepatocyte reparative changes and, in the exocrine pancreas, a mild decrease of cytoplasmic granules in the epithelium of the pancreatic acini. No histologic effects were seen in the endocrine pancreas.

In Study 2, the 25 mg/kg QD dosing regimen resulted in a 72.7% and 81.4% reduction in pancreas weight and tumor volume, respectively, while the 25 mg/kg 3×/wk dose group resulted in a 47.8% and 66.6% reduction. Dose-related decreases in pancreas weight and tumor volume (20.1% and 52.6%, respectively) were also observed at 15 mg/kg; no decreases were noted at 5 mg/kg. Median survival was 32 days with 25 mg/kg QD and 42 days with 25 mg/kg 3×/wk compared with 21 days in the control group.

In Study 3, treatment with GEM, (S,S)-(HO)2DEHSPM, and (S,S)-(HO)2DEHSPM+GEM resulted in 18.7%, 35.6%, and 42.4% decreases in body weight, respectively. Compared with controls, treatment with GEM, (S,S)-(HO)2DEHSPM, and GEM+(S,S)-(HO)2DEHSPM resulted in 24.7%, 58.8%, and 67.2% decreases in pancreas weight, and 37.8%, 58.4%, and 72.9% decreases in tumor volume, respectively, suggesting a synergistic or additive effect with combination treatment.

The incidence of liver metastasis was also decreased in all three treatment groups.

Conclusions:

(S,S)-(HO)2DEHSPM 25 mg/kg administered QD or 3×/wk inhibited the growth of human pancreatic ductal adenocarcinoma and prolonged survival in mice. Co-administration of (S,S)-(HO)2DEHSPM with gemcitabine appeared to have an additive or synergistic effect on reduction in the pancreatic tumor. These results support the clinical development of (S,S)-(HO)2DEHSPM for pancreatic cancer.

### Example 2—Evaluation of Human Pancreatic Cancer Cell Viability Following Culture with (S,S)-(HO)2DEHSPM in the Presence and Absence of GEM and NAB

Introduction:

(S,S)-(HO)2DEHSPM is an analogue of the native polyamine (PA) spermine. The PA uptake transport system is up regulated in a number of neoplasms including pancreatic ductal adenocarcinoma (PDA) suggesting a promising therapeutic target. This study evaluated the anti-proliferative effect of (S,S)-(HO)2DEHSPM in the presence and absence of gemcitabine (GEM) and nab-paclitaxel (NAB) in six human PDA cell lines.

Methods:

AsPC-1, BxPC-3, Capan-1, HPAF-II, MIA PaCa-2 and PANC-1 cells were seeded in 96-well plates and allowed to adhere overnight. After 24 h fresh media was added containing 0.5-10 μM concentrations of (S,S)-(HO)2DEHSPM alone or +0.5 μM GEM, +5 nM NAB, or + both, or containing GEM, NAB or GEM+NAB alone. Cell proliferation was measured in triplicate 24, 48, 72 and 96 h post-treatment and IC50 values were calculated.

Results:

(S,S)-(HO)2DEHSPM produced an anti-proliferative effect in all cell lines; maximal inhibition most often occurred with 10 μM. At 96 h, maximum mean inhibition with 10 μM (S,S)-(HO)2DEHSPM+GEM+NAB compared with GEM+NAB was 97.3% vs. 38.5% (BxPC-3), 90.1% vs. 47.1% (Capan-1), 89.7% vs. 38.3% (ASPC-1) and 39.4% vs. 8.0% (MIA PaCa-2) (p<0.005). (S,S)-(HO)2DEHSPM alone produced greater inhibition than GEM+NAB in ASPC-1, BxPC-3 and Capan-1 cells (p<0.005). In most cell lines IC50 decreased with (S,S)-(HO)2DEHSPM as treatment duration increased and combination treatments with (S,S)-(HO)2DEHSPM resulted in a further decrease in IC50, indicating an additive or synergistic effect with GEM and NAB on the decrease in cell viability.

Conclusion:

(S,S)-(HO)2DEHSPM, both alone and in combinations with GEM and NAB, exhibited a marked anti-proliferative effect against PDA. (S,S)-(HO)2DEHSPM alone and (S,S)-(HO)2DEHSPM+GEM+NAB were more effective than GEM+NAB, the current standard of care. These results confirm the anti-neoplastic potential of (S,S)-(HO)2DEHSPM and offer a rationale for its further investigation as a treatment for human pancreatic cancer.

### Example 3—Phase I Safety Study of (S,S)-(HO)2DEHSPM a Polyamine Metabolic Inhibitor, for Pancreatic Ductal Adenocarcinoma (PDA)

Background:

(S,S)-(HO)2DEHSPM (diethyl dihydroxyhomospermine), a polyamine (PA) analogue of spermine, inhibited growth in 6 human PDA cell lines and 3 murine xenograft tumor models. A Phase 1 dose escalation study assessed the safety, tolerability and pharmacokinetics (PK) of (S,S)-(HO)2DEHSPM in previously treated patients with locally advanced or metastatic PDA.

Methods:

In a modified 3+3 dose escalation scheme, daily subcutaneous injections of (S,S)-(HO)2DEHSPM were dosed at 0.05, 0.1, 0.2, 0.4 or 0.8 mg/kg, Monday-Friday for 3 weeks, followed by 5 weeks of observation (1 cycle), for 1 or 2 cycles. Safety and tolerability were evaluated by clinical and laboratory assessments. PK was evaluated on Days 1 and 18 of cycle 1. Efficacy was assessed by RECIST criteria and overall survival.

Results:

Twenty-nine patients were enrolled in 5 cohorts: (1: N=3; 2: N=5; 3: N=4; 4: N=7; 5: N=10). Twenty-six had ≥2 prior chemotherapy regimens. Drug-related toxicity in cohorts 1-4 was minimal, although one patient in cohort 4 developed focal pancreatitis at the site of the tumor at 2.3 months. Most common adverse events (AEs) were abdominal pain, constipation, decreased appetite, dehydration, diarrhea, fatigue, nausea and vomiting. Most were grades 1 or 2 or considered unlikely or not related. No drug-related bone marrow suppression or peripheral neuropathy was seen at any dose. No DLTs occurred in cohorts 1-4. Three patients in cohort 5 developed serious adverse events considered dose limiting toxicities: bacterial sepsis with metabolic acidosis (N=1), hepatic and renal failure with elevated lipase (N=1) and superior mesenteric vein thrombosis with metabolic acidosis (N=1). Plasma Cₘₐₓ and AUC₀₋ₜ increased linearly with dose. Stable disease occurred in 2 patients each in cohorts 3 and 4, and in 4 patients in cohort 5. Median survival in cohort 3 was 5.9 months.

Conclusions:

(S,S)-(HO)2DEHSPM was well tolerated in this study at dose levels 1-4; 0.8 mg/kg exceeded the maximum tolerated dose (MTD). Best tumor response and survival occurred with 0.2 mg/kg/day. The low incidence of AEs below the MTD and absence of bone marrow toxicity or peripheral neuropathy suggest the potential for (S,S)-(HO)2DEHSPM as an addition to front line treatment for PDA and justify a combination study.

### Example 4—Phase 1a/1b Safety Study of (S,S)-(HO)2DEHSPM in Combination with Gemcitabine and Nab-Paclitaxel as First Line Treatment for Subjects with Metastatic PDA

### Abstract

Background: (S,S)-(HO)2DEHSPM, a polyamine metabolic inhibitor, inhibited growth in 6 human pancreatic ductal adenocarcinoma (PDA) cell lines and 3 murine xenograft tumor models of human PDA. (S,S)-(HO)2DEHSPM monotherapy in heavily pre-treated PDA patients (>2 prior regimens, N=4) showed a median survival of 5.9 months at the optimal dose level.

Purpose: To assess the safety, tolerability, PK, and efficacy of (S,S)-(HO)2DEHSPM in combination with gemcitabine (G) and nab-paclitaxel (A) in patients with previously untreated metastatic PDA.

Methods: In a modified 3+3 dose escalation scheme, subcutaneous injections of (S,S)-(HO)2DEHSPM were dosed at 0.2, 0.4 or 0.6 mg/kg days 1-5 of each 28-day cycle. G (1000 mg/m²) and A (125 mg/m²) were administered intravenously on Days 1, 8, and 15 of each cycle. Safety and tolerability were evaluated by clinical and laboratory assessments. PK was evaluated on day 1 of cycle 1. Efficacy was assessed by CA19-9 levels, objective response was assessed by RECIST criteria, progression-free survival (PFS) and overall survival (OS).

Interim Results: Fifteen patients have been enrolled in 3 cohorts (1: N=4, 2: N=7, 3: N=4) and received up to 6 cycles of treatment (7 subjects are ongoing in cohorts 2 and 3). The most common adverse events related to (S,S)-(HO)2DEHSPM are fatigue (N=4), nausea (N=2) and injection site pain (N=2). There is no evidence of (S,S)-(HO)2DEHSPM-related bone marrow suppression or peripheral neuropathy. One patient in cohort 2 developed grade 3-4 reversible liver enzyme elevation. PK parameters in cohort 1 were below the limits of detection at most time points, but plasma Cₘₐₓ and AUC₀₋ₜ were measurable in cohorts 2 and 3. In those cohorts, CA19-9 levels decreased 76-95% in 7 of 8 evaluable subjects (1 additional subject TBD), with 5 patients achieving partial responses (4 ongoing) and 1 achieving stable disease. Median PFS and OS have not yet been reached.

Conclusions: Preliminary results suggest (S,S)-(HO)2DEHSPM is well tolerated when administered with G and A. Signals of efficacy support continued development of (S,S)-(HO)2DEHSPM in combination first-line treatment for PDA.

### Introduction

Polyamines (PAs) are aliphatic cations found in nearly all living cells, and they are critical for cell growth, protein synthesis and apoptosis. Although their concentrations are tightly controlled in normal cells, many tumors, including PDA, have elevated PA levels making them a promising therapeutic target. (S,S)-(HO)2DEHSPM, an analogue of the naturally occurring PA, spermine, is a polyamine metabolic inhibitor (PMI) that reduces PA pools by inhibiting key synthetic enzymes. Non-clinical studies showed (S,S)-(HO)2DEHSPM to have efficacy against PDA in vitro and in vivo, and a first-in-human monotherapy study in heavily pretreated patients with metastatic PDA (most had ≥2 prior chemotherapy regimens) demonstrated an acceptable safety profile below the MTD. In that study there was no significant bone marrow suppression or peripheral neuropathy as is commonly seen with gemcitabine (G) and nab-paclitaxel (A), suggesting the feasibility of (S,S)-(HO)2DEHSPM as an addition to combination first-line treatment.

### Study Design

This is a multicenter, open label, Phase 1a/1b study to evaluate to evaluate the safety, tolerability, pharmacokinetics and efficacy of (S,S)-(HO)2DEHSPM when administered in combination with G and A as first-line therapy in pancreatic cancer patients previously untreated for metastatic disease. The objective was to determine a recommended Phase 2 dose. Using a modified 3+3 dose escalation scheme, cohorts of subjects were dosed with subcutaneous injections of (S,S)-(HO)2DEHSPM at 0.2, 0.4 or 0.6 mg/kg days 1-5 of each 28-day cycle. Gemcitabine (1000 mg/m²) and A (125 mg/m²) were administered intravenously on Days 1, 8, and 15 of each cycle. Safety and tolerability were evaluated by clinical and laboratory assessments. PK was evaluated on day 1 of cycle 1. Efficacy was assessed by objective response rate (ORR) using RECIST criteria and by changes in CA19-9 levels. Subjects were treated until disease progression or the development of dose-limiting toxicity. Based upon initial safety findings and preliminary signals of efficacy, the protocol was amended to evaluate progression-free survival (PFS) and overall survival (OS) and expand the study to up to 36 subjects at the recommended Phase 2 dose. To date, 20 subjects were enrolled in cohorts 1-3 and evaluated for dose limiting toxicity and early signals of efficacy.

### Demographics

Table 1 shows the demographics of the study population. There were no significant differences in gender or age between cohorts. Most of the subjects were white.

TABLE 1
Demographics

| | Cohort 1 | | Cohort 2 | | Cohort 3 | | | |
|---|---|---|---|---|---|---|---|---|
| | (0.2 mg/kg) | | (0.4 mg/kg) | | (0.6 mg/kg) | | All Cohorts | |
| | (N = 4) | | (N = 7) | | (N = 9) | | (N = 20) | |
| Age (years)* | | | | | | | | |
| Mean (SD) | 66.8 | (9.88) | 62.1 | (9.55) | 65.8 | (7.66) | 64.7 | (8.53) |
| Median (Range) | 71 | (52-73) | 65 | (42-72) | 68 | (47-74) | 66 | (42-74) |
| Gender n (%) | | | | | | | | |
| Male | 2 | (50.0%) | 4 | (57.1%) | 4 | (44.4%) | 10 | (50.0%) |
| Female | 2 | (50.0%) | 3 | (42.9%) | 5 | (55.6%) | 10 | (50.0%) |
| Race n (%) | | | | | | | | |
| White | 3 | (75.0%) | 7 | (100.0%) | 8 | (88.9%) | 18 | (90.0%) |

| | | | | | | | |
|---|---|---|---|---|---|---|---|
| Asian | 1 | (25.0%) | 0 | 1 | (11.1%) | 2 | (10.0%) |

Table 2 shows that the pharmacokinetic parameters for (S,S)-(HO)2DEHSPM in cohort 1 were below the limits of detection at most time points, but plasma Cₘₐₓ and Tₘₐₓ were measurable. Cₘₐₓ values were similar to the previous Phase 1 monotherapy study described and increased linearly with dose. Tₘₐₓ was the same in both studies.

TABLE 2
Pharmacokinetics

| | Cohort 1 | Cohort 2 | Cohort 3 |
|---|---|---|---|
| | (0.2 mg/kg) | (0.4 mg/kg) | (0.6 mg/kg) |
| | (N = 4) | (N = 7) | (N = 5*) |
| Cₘₐₓ (μg/mL) | | | |
| Mean | 0.0266 | 0.1147 | 0.1467 |
| Range | 0.0132-0.0416 | 0.0771-0.167 | 0.0919-0.195 |
| Tₘₐₓ (hr) | | | |
| Mean | 0.5 | 0.5 | 0.5 |

*PK samples were collected for 5 of the 9 patients in Cohort 3.

### Safety

Table 3A shows (S,S)-(HO)2DEHSPM-related Adverse Events Occurring in ≥2 Subjects (10%), N=20*

TABLE 3A

| | | | | | | Total |
|---|---|---|---|---|---|---|
| Event | Grade 1 | Grade 2 | Grade 3 | Grade 4 | Grade 5 | N (%) |
| Fatigue | 4 | 3 | 1 | 0 | 0 | 8 (40%) |
| Elevated Liver | 0 | 0 | 3 | 1 | 0 | 4 (20%) |
| Function Tests | | | | | | |
| (LFTs) | | | | | | |
| Injection site | 4 | 0 | 0 | 0 | 0 | 4 (20%) |
| pain | | | | | | |
| Diarrhea | 1 | 1 | 0 | 0 | 0 | 2 (10%) |
| Nausea | 1 | 1 | 0 | 0 | 0 | 2 (10%) |

*The Safety Population includes all subjects who received at least one dose of (S,S)-(HO)2DEHSPM (N = 20). Related events were defined as definitely, probably or possibly related and not related events as unlikely or not related. In the total N, subjects are counted only once at the highest grade for each event.

Table 3B shows the frequency of Grade ≥3 Adverse Events of Special Interest, N=20 and Comparison to G+A Historical Control Data*

TABLE 3B

| | | | |
|---|---|---|---|
| Grade ≥ 3 Adverse Events of Interest | N | % | G + A %* |

Hematologic Events

| | | | |
|---|---|---|---|
| Leukopenia (G3-4) | 3 | 15% | 31% |
| Neutropenia (G3-4) | 6 | 30% | 38% |
| Anemia | 3 | 15% | 13% |
| Thrombocytopenia | 1 | 5% | 17% |

Non-hematologic Events

| | | | |
|---|---|---|---|
| Peripheral Neuropathy | 0 | 0% | 17% |
| Fatigue | 1 | 5% | 17% |
| Diarrhea | 0 | 0% | 6% |

*Historical control data, MPACT study, G + A arm, N = 431
Source: Von Hoff 2013 NEJM

Table 3C shows Grade ≥3 Adverse Events Related to Any Study Medication, N=20

TABLE 3C

| Event | (S,S)-(HO)2DEHSPM | G + A | All 3 | Total N (%) | |
|---|---|---|---|---|---|
| Neutropenia | 0 | 6 | 0 | 6 | (30%) |
| Elevated LFTs | 4 | 1 (G) | 0 | 4 | (20%) |
| Anemia | 0 | 3 | 0 | 3 | (15%) |
| Fatigue | 0 | 0 | 1 | 1 | (5%) |
| Elevated Lipase | 1 | 0 | 0 | 1 | (5%) |
| Thrombocytopenia | 0 | 2 | 0 | 1 | (5%) |
| Dyspnea | 0 | 0 | 1 | 1 | (5%) |
| Delirium | 0 | 1 (A) | 0 | 1 | (5%) |
| Hiccups (Intractable) | 0 | 1 (A) | 0 | 1 | (5%) |
| Hyponatremia | 0 | 0 | 1 | 1 | (5%) |
| Dehydration | 0 | 1 | 0 | 1 | (5%) |

The most common Grade≥3 AEs related to any study medication were neutropenia in 6 subjects attributed to G+A, and elevated liver function tests (LFT) in 4 subjects attributed to (S,S)-(HO)2DEHSPM, one of which was also attributed to G. (S,S)-(HO)2DEHSPM-related increases in liver toxicity occurred after several cycles of treatment, was asymptomatic in all but one subject, and reversed in all but one subject when (S,S)-(HO)2DEHSPM administration was interrupted and dose-reduced or discontinued.

### Safety Summary:

The addition of (S,S)-(HO)2DEHSPM to the treatment regimen did not increase the frequency of Grade ≥3 hematologic events or peripheral neuropathy, fatigue or diarrhea when compared with historical control data on G+A combination therapy.

### Conclusions:

(S,S)-(HO)2DEHSPM was well-tolerated when administered at doses tested in combination with G+A in subjects with previously untreated metastatic pancreatic adenocarcinoma. Treatment-related liver function abnormalities were mostly asymptomatic and mostly reversed when (S,S)-(HO)2DEHSPM was interrupted and dose-reduced or discontinued. For example, a rescue dosing regimen was initiated for any Grade ≥3 liver function toxicities, similar to that implemented for Grade ≥3 hematologic toxicity events wherein dosing is interrupted entirely or decreased for a period of time and then resumed at half dose for a period of time when liver function abnormalities return to baseline or normal.

There was no evidence that (S,S)-(HO)2DEHSPM potentiates the Grade ≥3 hematologic events, peripheral neuropathy typically seen with G+A alone. ORR (62%) and DCR (85%) exceeded the historical rates reported for G+A (23%, 48%) and FOLFIRINOX (32%, 70%) in pivotal trials; responses were accompanied by large decreases in CA19-9 levels.

The data supports (S,S)-(HO)2DEHSPM alone or in combination with G+A as a suitable first-line treatment for advanced PDA.

### Example 5—Improving Patient Safety Profiles and Limiting Liver Toxicity

As part of the same study described in Example 4, one cohort of patients (Cohort 4) was administered a combination shortened daily dosing regimen/periodic dosing regimen and liver toxicities were compared to the reference dosing regimen described in Example 4.

Cohort 4 was administered 0.4 mg/kg/day of (S,S)-(HO)2DEHSPM for 5 consecutive days (for a total of 5 doses/cycle) beginning on day 1 (i.e. days 1-5) of each of Cycles 1 and 2. Thereafter Cohort 4 was administered 0.4 mg/kg/day of (S,S)-(HO)2DEHSPM on Days 1, 8 and 15 beginning on day 1 of Cycle 3 (for a total of 3 doses per cycle) and continuing for each cycle thereafter. Cohort 4 patients were treated for 3 to 6 or more total cycles depending on the individual patient's tolerance of treatment.

The results show that severe liver toxicities are unexpectedly reduced or eliminated as compared to the cohorts of Example 4 and particularly as compared to Cohort 3 of Example 4. Based upon the ability to manage liver toxicity in Cohort 4 to <Grade 3, the dose and regimen used in Cohort 4 was recommended as the chosen dose and regimen for further development.

The patent and scientific literature referred to herein establishes the knowledge that is available to those with skill in the art. All United States patents and published or unpublished United States patent applications cited herein are incorporated by reference. All published foreign patents and patent applications cited herein are hereby incorporated by reference. All other published references, documents, manuscripts and scientific literature cited herein are hereby incorporated by reference.

While this invention has been particularly shown and described with references to preferred embodiments thereof, it will be understood by those skilled in the art that various changes in form and details may be made therein without departing from the scope of the invention encompassed by the appended claims. It should also be understood that the embodiments described herein are not mutually exclusive and that features from the various embodiments may be combined in whole or in part in accordance with the invention.

1. A method for treating cancer in a patient comprising administering to a patient in need thereof, a dosing regimen of (S,S)-(HO)2DEHSPM, wherein the dosing regimen comprises administering (S,S)-(HO)2DEHSPM daily for 5 consecutive days of each treatment cycle for no more than two 4 consecutive treatment cycles wherein each treatment cycle is about 28 days.

2. The method of claim 1, wherein the dosing regimen comprises administering (S,S)-(HO)2DEHSPM daily for 5 consecutive days for no more than two consecutive treatment cycles.

3. The method of claim 1, wherein the daily free base equivalent dose of (S,S)-(HO)2DEHSPM is selected from about 0.14 mg/kg/day, about 0.27 mg/kg/day, and about 0.41 mg/kg/day.

4-7. (canceled)

8. The method of claim 1, further comprising co-administering gemcitabine (GEM) or nab-paclitaxel (NAB) or both GEM and NAB (GEM/NAB) with (S,S)-(HO)2DEHSPM for at least one treatment cycle.

9. The method of claim 8, wherein co-administration comprises administering gemcitabine (GEM) or nab-paclitaxel (NAB) or both GEM and NAB (GEM/NAB) on days 1, 8 and 15 of at least one treatment cycle.

10. (canceled)

11. The method of claim 10, further comprising administering (S,S)-(HO)2DEHSPM periodically during a treatment cycle subsequent to treatment cycle 2 wherein periodic administration comprises no more than 14 doses of (S,S)-(HO)2DEHSPM administered on non-consecutive days.

12-13. (canceled)

14. A method for treating cancer in a patient in need thereof comprising administering to the patient, a dosing regimen of (S,S)-(HO)2DEHSPM wherein the dosing regimen comprises administering (S,S)-(HO)2DEHSPM daily for 5 consecutive days during the first week of each treatment cycle for two consecutive treatment cycles, wherein each treatment cycle is about 28 days, followed by administering (S,S)-(HO)2DEHSPM periodically on days 1, 8 and 15 during the third and subsequent treatment cycles.

15. The method of claim 14 wherein the dosing regimen comprises administering (S,S)-(HO)2DEHSPM daily for 5 consecutive days at a free base equivalent dose of about 0.27 mg/kg/day during the first week of each of the first two treatment cycles, followed by administering (S,S)-(HO)2DEHSPM at a free base equivalent dose of about 0.21 mg/kg/day to about 0.34 mg/kg/day on days 1, 8 and 15 of the third and subsequent treatment cycles.

16. (canceled)

17. The method of claim 15, wherein treatment is continued until the total cumulative free base equivalent dose is about 8.2 mg/kg or less.

18. The method of claim 17, wherein treatment is continued until the total cumulative free base equivalent dose is about 3.4 mg/kg to about 6.9 mg/kg.

19. The method of claim 17, wherein treatment is continued until the total cumulative free base equivalent dose is about 5.8 mg/kg to about 6.5 mg/kg.

20. (canceled)

21. The method of claim 14, further comprising co-administering GEM/NAB during each of the treatment cycles.

22. The method of claim 21, wherein GEM/NAB is administered on days 1, 8 and 15 of each treatment cycle.

23. (canceled)

24. The method of claim 14, wherein the free base equivalent dose of (S,S)-(HO)2DEHSPM is selected from 0.14 mg/kg/day, 0.27 mg/kg/day, and 0.41 mg/kg/day.

25-30. (canceled)

31. A method of treating cancer in a patient comprising administering to a patient in need thereof no more than 14 doses of (S,S)-(HO)2DEHSPM per each treatment cycle wherein dosing occurs on non-consecutive days and wherein each treatment cycle is 28 days.

32. The method of claim 31, wherein the patient is administered no more than 10 doses per treatment cycle.

33. (canceled)

34. The method of claim 31, wherein the patient is administered (S,S)-(HO)2DEHSPM on days 1, 8 and 15 of each treatment cycle.

35. (canceled)

36. The method of claim 1, wherein the (S,S)-(HO)2DEHSPM is administered as a subcutaneous injection.

37. The method of claim 1, wherein the (S,S)-(HO)2DEHSPM is administered as (S,S)-(HO)2DEHSPM.4HCl.

38. (canceled)
